SEQUENCE LISTING (64 sequences)

SEQ ID NO: 1 - protein, 6 aa, synthetic construct
SSYYMA

SEQ ID NO: 2 - protein, 5 aa, synthetic construct
DSYMS

SEQ ID NO: 3 - protein, 17 aa, synthetic construct
DMYPDNGDSSYNQKFRE

SEQ ID NO: 4 - protein, 8 aa, synthetic construct
APRWYFSV

SEQ ID NO: 5 - protein, 11 aa, synthetic construct
RASQDISNYLN

SEQ ID NO: 6 - protein, 7 aa, synthetic construct
YTSRLRS

SEQ ID NO: 7 - protein, 9 aa, synthetic construct
QQGHTLPPT

SEQ ID NO: 8 - protein, 117 aa, synthetic construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 9 - protein, 107 aa, synthetic construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 10 - protein, 5 aa, synthetic construct
NYLIE

SEQ ID NO: 11 - protein, 17 aa, synthetic construct
VINPGSGDTYYSEKFKG

SEQ ID NO: 12 - protein, 5 aa, synthetic construct
DRLDY

SEQ ID NO: 13 - protein, 11 aa, synthetic construct
HASQDISSYIV

SEQ ID NO: 14 - protein, 7 aa, synthetic construct
HGTNLED

SEQ ID NO: 15 - protein, 9 aa, synthetic construct
VHYAQFPYT

SEQ ID NO: 16 - protein, 114 aa, synthetic construct
EVQLVQSGAEVKKPGASVKVSCKASGYAFTNYLIEWVRQAPGQGLEWIGVINPGSGDTYYSEKFKGRVTITRDTSTSTAYLELSSLRSEDTAVYYCARDRLDYWGQGTLVTVSS

SEQ ID NO: 17 - protein, 107 aa, synthetic construct
DIQMTQSPSSLSASVGDRVTITCHASQDISSYIVWYQQKPGKAPKLLIYHGTNLEDGVPSRFSGSGSGTDFTLTISSLQPEDFATYYCVHYAQFPYTFGQGTKVEIK

SEQ ID NO: 18-25 - skipped (8 entries)

SEQ ID NO: 26 - protein, 17 aa, synthetic construct
AIFTGSGAEYKAEWAKG

SEQ ID NO: 27 - protein, 13 aa, synthetic construct
DAGYDYPTHAMHY

SEQ ID NO: 28 - protein, 11 aa, synthetic construct
RASQGISSSLA

SEQ ID NO: 29 - protein, 7 aa, synthetic construct
GASETES

SEQ ID NO: 30 - protein, 12 aa, synthetic construct
QNTKVGSSYGNT

SEQ ID NO: 31 - protein, 123 aa, synthetic construct [sequence omitted]

SEQ ID NO: 32 - protein, 110 aa, synthetic construct [sequence omitted]

SEQ ID NO: 33 - DNA, 30 bp, synthetic construct
  source 1..30: mol_type = other DNA
cttccactttcatttcacaaactaattttt

SEQ ID NO: 34 - DNA, 30 bp, synthetic construct; identical to SEQ ID NO: 33
  source 1..30: mol_type = other DNA

SEQ ID NO: 35 - DNA, 30 bp, synthetic construct
  source 1..30: mol_type = other DNA
agagccacagttgatatgctatctattaag

SEQ ID NO: 36 - DNA, 30 bp, synthetic construct
  source 1..30: mol_type = other DNA
agccacagtttgatatgctatttattaggg

SEQ ID NO: 37 - DNA, 66 bp, synthetic construct
  source 1..66: mol_type = other DNA
agtaccgaacttattagcatgctgttcttccactttcatttcacaaactaatttttcaggagcagc

SEQ ID NO: 38 - DNA, 66 bp, synthetic construct; identical to SEQ ID NO: 37
  source 1..66: mol_type = other DNA

SEQ ID NO: 39 - DNA, 66 bp, synthetic construct
  source 1..66: mol_type = other DNA
aggtgctcctctttctgtgcacatcagagccacagtttgatatgctatttattagggttgctgccc

SEQ ID NO: 40 - DNA, 66 bp, synthetic construct
  source 1..66: mol_type = other DNA
aggtgctcctctttctgtgcacatcagagccacagttgatatgctatctattaaggttgctgtcaa

SEQ ID NO: 41 - DNA, 75 bp, synthetic construct
  source 1..75: mol_type = other DNA
ttattagcatgctgttcttccactttcatttcacaaactaatttttcaggagcagcagagacaagtgtcacctaa

SEQ ID NO: 42 - DNA, 75 bp, synthetic construct
  source 1..75: mol_type = other DNA
  misc_difference 26: note = a, c, t, g, unknown or other
ctttctgtgcacatcagagccacagnttgatatgctatctattaaggttgctgtcaagcctccatggagagtacc

SEQ ID NO: 43 - DNA, 72 bp, synthetic construct
  source 1..72: mol_type = other DNA
ctttctgtgcacatcagagccacagatatgctatctattaaggttgctgtcaagcctccatggagagtaccg

SEQ ID NO: 44 - DNA, 71 bp, synthetic construct
  source 1..71: mol_type = other DNA
ctttctgtgcacatcagagccacagtatgctatctattaaggttgctgtcaagcctccatggagagtaccg

SEQ ID NO: 45 - DNA, 74 bp, synthetic construct
  source 1..74: mol_type = other DNA
ctttctgtgcacatcagagccacattgatatgctatctattaaggttgctgtcaagcctccatggagagtaccg

SEQ ID NO: 46 - DNA, 75 bp, synthetic construct
  source 1..75: mol_type = other DNA
ctttctgtgcacatcagagccacagttgatatgctatctattaaggttgctgtcaagcctccatggagagtaccg

SEQ ID NO: 47 - DNA, 73 bp, synthetic construct
  source 1..73: mol_type = other DNA
ctttctgtgcacatcagagccacttgatatgctatctattaaggttgctgtcaagcctccatggagagtaccg

SEQ ID NO: 48 - DNA, 73 bp, synthetic construct
  source 1..73: mol_type = other DNA
ctttctgtgcacatcagagccacatgatatgctatctattaaggttgctgtcaagcctccatggagagtaccg

SEQ ID NO: 49 - DNA, 75 bp, synthetic construct
  source 1..75: mol_type = other DNA
  misc_difference 26: note = a, c, t, g, unknown or other
  misc_difference 27: note = a, c, t, g, unknown or other
ctttctgtgcacatcagagccacagnnttgatatgctatctattaaggttgctgtcaagcctccatggagagtac

SEQ ID NO: 50 - DNA, 72 bp, synthetic construct
  source 1..72: mol_type = other DNA
ctttctgtgcacatcagagccactgatatgctatctattaaggttgctgtcaagcctccatggagagtaccg

SEQ ID NO: 51 - DNA, 61 bp, synthetic construct
  source 1..61: mol_type = other DNA
ctttctgtgcacatcagagccacatattaaggttgctgtcaagcctccatggagagtaccg

SEQ ID NO: 52 - DNA, 61 bp, synthetic construct
  source 1..61: mol_type = other DNA
ctttctgtgcacatcagagccacagattaaggttgctgtcaagcctccatggagagtaccg

SEQ ID NO: 53 - DNA, 23 bp, unidentified
  source 1..23: mol_type = unassigned DNA
tccaaaactctatcaaaaccggg

SEQ ID NO: 54 - DNA, 23 bp, unidentified
  source 1..23: mol_type = unassigned DNA
tcttacctctgtattcacttagg

SEQ ID NO: 55 - DNA, 23 bp, unidentified
  source 1..23: mol_type = unassigned DNA
gaagcctaaactgatgtaccagg

SEQ ID NO: 56 - DNA, 23 bp, unidentified
  source 1..23: mol_type = unassigned DNA
cagcaacacctcagtcagcgagg

SEQ ID NO: 57 - DNA, 23 bp, unidentified
  source 1..23: mol_type = unassigned DNA
agagaggttccgccacacaaagg

SEQ ID NO: 58 - DNA, 23 bp, unidentified
  source 1..23: mol_type = unassigned DNA
accgaaatgatcaggtactgggg

SEQ ID NO: 59 - DNA, 23 bp, unidentified
  source 1..23: mol_type = unassigned DNA
ctgctgtaaccccataagcatgg

SEQ ID NO: 60 - DNA, 23 bp, unidentified
  source 1..23: mol_type = unassigned DNA
ggaagccaagaagaagaaggagg

SEQ ID NO: 61 - DNA, 23 bp, unidentified
  source 1..23: mol_type = unassigned DNA
atcccgggacacagacacaaagg

SEQ ID NO: 62 - DNA, 23 bp, unidentified
  source 1..23: mol_type = unassigned DNA
cagagtttgaccgcctcccaagg

SEQ ID NO: 63 - DNA, 23 bp, unidentified
  source 1..23: mol_type = unassigned DNA
atccagcagtcaatgataacagg

SEQ ID NO: 64 - DNA, 23 bp, unidentified
  source 1..23: mol_type = unassigned DNA
cagcttagcaccttcggtcaggg

(19) United States
(12) Patent Application Publication
(10) Pub. No.: US 2023/0374497 A1
(43) Pub. Date: Nov. 23, 2023

(54) CRISPR/Cas9 MULTIPLEX KNOCKOUT OF HOST CELL PROTEINS

(71) Applicants: GENENTECH, INC., South San Francisco, CA (US); HOFFMANN-LA ROCHE INC., Little Falls, NJ (US)

(72) Inventors: Amy SHEN, South San Francisco, CA (US); Inn Huam Yuk, South San Francisco, CA (US); Peggy Wai No Ko, South San Francisco, CA (US); Shahram Misaghi, South San Francisco, CA (US); Simon Thomas Auslaender, Penzberg (DE); Midori Greenwood-Goodwin, South San Francisco, CA (US); Michael Wilson Laird, South San Francisco, CA (US); Benedikt Alois Claudius Oswald, Penzberg, DE (US)

(73) Assignees: GENENTECH, INC., South San Francisco, CA (US); HOFFMANN-LA ROCHE INC., Little Falls, NJ (US)

(21) Appl. No.: 18/176,062

(22) Filed: Feb. 28, 2023

Related U.S. Application Data
(63) Continuation of application No. PCT/US2021/048046, filed on Aug. 27, 2021.
(60) Provisional application No. 63/071,764, filed on Aug. 28, 2020.

(51) Int. Cl. C12N 15/10; C12N 15/11; C12N 9/22; C12N 15/90; C12N 5/00

(52) U.S. Cl. CPC C12N 15/1082; C12N 15/11; C12N 9/22; C12N 15/907; C12N 5/00; C12N 2310/20; C12N 2800/80

(57) ABSTRACT

The present disclosure relates to modified mammalian cells having reduced or eliminated expression of certain cellular proteins, CRISPR/Cas9 multiplex knockout strategies for making such cells, and methods of using such cells, e.g., in the context of cell-based therapy or as host cells in the production of a product of interest.

### CROSS-REFERENCE TO RELATED APPLICATIONS

This application is a continuation application of International Patent Application No. PCT/US2021/048046, filed Aug. 27, 2021, which claims priority to U.S. Provisional Application No. 63/071,764, filed Aug. 28, 2020, the contents of which are incorporated by reference in its entirety, and to which priority is claimed.

### SEQUENCE LISTING

The instant application contains a Sequence Listing which has been submitted in XML format via EFS-Web and is hereby incorporated by reference in its entirety. Said XML copy, created on Jul. 6, 2023, is named 00B206_1323.xml and is 54,157 bytes in size.

### 1. FIELD OF INVENTION

The present disclosure relates to modified mammalian cells having reduced or eliminated expression of certain cellular proteins, CRISPR/Cas9 multiplex knockout strategies for making such cells, and methods of using such cells, e.g., in the context of cell-based therapy or as host cells in the production of a product of interest.

### 2. BACKGROUND

Despite advances made in manufacturing of therapeutic proteins in Chinese hamster ovary (CHO) cells in the last decades (Lalonde et al., Journal of Biotechnology. 2017;251:128-140 and Kunert et al., Appl Microbiol Biotechnol. 2016;100(8):3451-3461), economic incentives to further increase productivity, improve stability, and engineer specific traits remain strong. (Wells et al., Biotechnology Journal. 2017;12(1):1600105). The emergence of clustered regularly interspaced short palindromic repeat (CRISPR)/Cas9 systems for gene editing and recently developed robust proteomics methods have revolutionized cell line engineering. Such genetic manipulations have been used to reduce apoptosis (Baek et al., Heterologous Protein Production in CHO Cells. Springer; 2017:71-85), eliminate antibody fucosylation (Grav et al., Biotechnology Journal. 2015;10(9):1446-1456), improve drug product stability (Chiu et al., Biotechnology and Bioengineering. 2017;114(5):1006-1015 and Laux et al., Biotechnology and Bioengineering. 2018;115(10):2530-2540), improve CHO cell secretory pathway (Kol et al., Nature Communications. 2020;11(1):1-10), and reduce CHO host cell protein levels. (Walker et al., MAbs. Vol 9. Taylor & Francis; 2017:654-663).

CRISPR/Cas9 protocols that utilize DNA plasmids to deliver Cas9 and gRNA to generate knockouts (Amann et al., Deca CHO KO: exploring the limitations of CRISPR/Cas9 multiplexing in CHO cells. Design of Optimal CHO Protein N-glycosylation Profiles 2018:36; Grav et al., Heterologous Protein Production in CHO Cells. Springer; 2017:101-118; and Sergeeva et al., CRISPR Gene Editing. Springer; 2019:213-232) have several shortcomings. The wide range in editing efficiency of different gRNA sequences necessitates a lengthy and expensive process of synthesizing, cloning, and screening various gRNA plasmids. Targeted NGS, which is the gold standard for quantifying CRISPR edits, is resource-intensive and expensive while other screening approaches such as western blot analysis, T7 endonuclease I assays, and size-based PCR amplicon analysis (VanLeuven et al., Biotechniques. 2018;64(6):275-278) lack speed, sensitivity, and ability to accurately differentiate weak gRNAs from more efficient ones. (Sentmanat et al., Scientific Reports. 2018;8(1):1-8). Furthermore, efficient stable integration of the transfected Cas9 DNA (Lino et al., Drug Deliv. 2018;25(1):1234-1257) can have undesirable outcomes for engineered CHO cell lines for manufacturing therapeutic proteins. Thus, there remains a need in the art for an efficient strategy to achieve multiplex knockouts.

### 3. SUMMARY

In certain embodiments, the present disclosure is directed to methods of producing a cell comprising edits at two or more target loci, wherein the method comprises combining two or more guide RNAs (gRNAs) capable of directing CRISPR/Cas9-mediated indel formation at respective target loci with Cas9 protein to form a ribonucleoprotein complex (RNP); serially transfecting a population of cells with the RNP until at least about 10% indel formation is achieved at each target locus; and isolating a cell comprising edits at two or more target loci by single cell cloning of the cell from the population of serially transfected cells. In certain embodiments, the gRNA is an sgRNA. In certain embodiments, the gRNA comprises a crRNA and a tracrRNA. In certain embodiments, the crRNA is an XT-gRNA.

In certain embodiments of the methods of producing a cell comprising edits at two or more target loci described herein, the population of cells is serially transfected with the RNP until at least about 20% indel formation is achieved at each target locus. In certain embodiments, the population of cells is serially transfected with the RNP until at least about 30% indel formation is achieved at each target locus. In certain embodiments, the population of cells is serially transfected with the RNP until at least about 40% indel formation is achieved at each target locus. In certain embodiments, the population of cells is serially transfected with the RNP until at least about 50% indel formation is achieved at each target locus. In certain embodiments, the population of cells is serially transfected with the RNP until at least about 60% indel formation is achieved at each target locus.

In certain embodiments of the methods of producing a cell comprising edits at two or more target loci described herein, the ratio of moles of RNP to number of transfected cells is between about 0.1 pmol per 10⁶ cells to about 5 pmol per 10⁶ cells. In certain embodiments, the ratio of moles of RNP to number of transfected cells is about 0.15 pmol per 10⁶ cells. In certain embodiments, the ratio of moles of RNP to number of transfected cells is about 0.17 pmol per 10⁶ cells. In certain embodiments, the ratio of moles of RNP to number of transfected cells is about 0.2 pmol per 10⁶ cells. In certain embodiments, the ratio of moles of RNP to number of transfected cells is about 1 pmol per 10⁶ cells. In certain embodiments, the ratio of moles of RNP to number of transfected cells is about 2 pmol per 10⁶ cells. In certain embodiments, the ratio of moles of RNP to number of transfected cells is about 3 pmol per 10⁶ cells.

In certain embodiments of the methods of producing a cell comprising edits at two or more target loci described herein, three or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus. In certain embodiments, four or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus. In certain embodiments, five or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus. In certain embodiments, six or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus. In certain embodiments, seven or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus. In certain embodiments, eight or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus. In certain embodiments, nine or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus. In certain embodiments, ten or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

In certain embodiments, the cell is a T cell, an NK cell, a B cell, a dendritic cell, a CHO cell, a COS-7 cell; an HEK 293 cell, a BHK cells, a TM4 cell, a CV1 cell; a VERO-76 cell; a HELA cells; or an MDCK cell.

In certain embodiments of the methods producing a cell comprising edits at two or more target loci described herein, the two or more gRNAs capable of directing CRISPR/Cas9-mediated indel formation at respective target loci are identified via a efficiency screen comprising: (a) transfecting a population of cells with a population of RNPs, where each RNP comprises a gRNA capable of directing CRISPR/Cas9-mediated indel formation at a target locus; and (b) sequencing the target loci to identify gRNAs based on their efficiency in directing CRISPR/Cas9-mediated indel formation. In certain embodiments, the sequencing is performed using Sanger sequencing.

In certain embodiments, the present disclosure is directed to a cell composition, wherein the cell comprises edits at two or more target loci, wherein the edits are the result of: combining two or more gRNAs capable of directing CRISPR/Cas9-mediated indel formation at respective target loci with Cas9 protein to form an RNP; serially transfecting a population of cells with the RNP until at least about 10% indel formation is achieved at each target locus; and isolating the cell comprising edits at two or more target loci by single cell cloning of the cell from the population of serially transfected cells

In certain embodiments, the present disclosure is directed to a host cell composition, wherein the host cell comprises: a nucleic acid encoding a non-endogenous polypeptide of interest; and edits at two more target loci, wherein the edits are the result of: combining two or more gRNAs capable of directing CRISPR/Cas9-mediated indel formation at respective target loci with Cas9 protein to form an RNP; serially transfecting a population of cells with the RNP until at least about 10% indel formation is achieved at each target locus; and isolating the host cell comprising edits at two or more target loci by single cell cloning of the host cell from the population of serially transfected cells.

In certain embodiments of the compositions disclosed herein, the gRNA is an sgRNA. In certain embodiments of the compositions disclosed herein, the gRNA comprises a crRNA and a tracrRNA. In certain embodiments of the compositions disclosed herein, the crRNA is an XT-gRNA. In certain embodiments of the compositions disclosed herein, the population of cells is serially transfected with the RNP until at least about 20% indel formation is achieved at each target locus. In certain embodiments, the population of cells is serially transfected with the RNP until at least about 30% indel formation is achieved at each target locus. In certain embodiments, the population of cells is serially transfected with the RNP until at least about 40% indel formation is achieved at each target locus. In certain embodiments, the population of cells is serially transfected with the RNP until at least about 50% indel formation is achieved at each target locus. In certain embodiments, the population of cells is serially transfected with the RNP until at least about 60% indel formation is achieved at each target locus.

In certain embodiments of the compositions disclosed herein, the ratio of moles of RNP to number of transfected cells is between about 0.1 pmol per 10⁶ cells to about 5 pmol per 10⁶ cells. In certain embodiments, the ratio of moles of RNP to number of transfected cells is about 0.15 pmol per 10⁶ cells. In certain embodiments, the ratio of moles of RNP to number of transfected cells is about 0.17 pmol per 10⁶ cells. In certain embodiments, the ratio of moles of RNP to number of transfected cells is about 0.2 pmol per 10⁶ cells. In certain embodiments, the ratio of moles of RNP to number of transfected cells is about 1 pmol per 10⁶ cells. In certain embodiments, the ratio of moles of RNP to number of transfected cells is about 2 pmol per 10⁶ cells. In certain embodiments, the ratio of moles of RNP to number of transfected cells is about 3 pmol per 10⁶ cells.

In certain embodiments of the compositions disclosed herein, the cell is a T cell, an NK cell, a B cell, a dendritic cell, a CHO cell, a COS-7 cell; an HEK 293 cell, a BHK cells, a TM4 cell, a CV1 cell; a VERO-76 cell; a HELA cells; or an MDCK cell.

In certain embodiments of the compositions disclosed herein, the two or more gRNAs capable of directing CRISPR/Cas9-mediated indel formation at respective target loci are identified via an efficiency screen comprising: transfecting a population of cells with a population of RNPs, where each RNP comprises a gRNA capable of directing CRISPR/Cas9-mediated indel formation at a target locus; and sequencing the target loci to identify gRNAs based on their efficiency in directing CRISPR/Cas9-mediated indel formation. In certain embodiments, the sequencing is performed using Sanger sequencing.

In certain embodiments, the methods for producing a polypeptide of interest described herein comprise: culturing a host cell composition comprising: a nucleic acid encoding a non-endogenous polypeptide of interest; and edits at two or more target loci, wherein the edits are the result of: (1) combining two or more gRNAs capable of directing CRISPR/Cas9-mediated indel formation at respective target loci with Cas9 protein to form an RNP; (2) serially transfecting a population of cells with the RNP until about 10% indel formation is achieved at each target locus; and (3) isolating the host cell comprising edits at two or more target loci by single cell cloning of the host cell from the population of serially transfected cells; and isolating the polypeptide of interest expressed by the cultured host cell.

In certain of the above described embodiments, the methods provided in the present disclosure further comprise purifying the product of interest, harvesting the product of interest, and/or formulating the product of interest.

In certain of the above described embodiments, the cell is a mammalian cell. In certain of the above described embodiments, the mammalian cell is a CHO cell.

In certain of the above described embodiments, the cell expresses a product of interest. In certain of the above described embodiments, the product of interest expressed by the mammalian cells is encoded by a nucleic acid sequence. In certain of the above described embodiments, the nucleic acid sequence is integrated in the cellular genome of the mammalian cells at a targeted location. In certain of the above described embodiments, the product of interest expressed by the cells is further encoded by a nucleic acid sequence that is randomly integrated in the cellular genome of the mammalian cells.

In certain of the above described embodiments, the product of interest comprises a protein. In certain of the above described embodiments, the product of interest comprises a recombinant protein. In certain of the above described embodiments, the product of interest comprises an antibody or an antigen-binding fragment thereof. In certain of the above described embodiments, the antibody is a multispecific antibody or an antigen-binding fragment thereof. In certain of the above described embodiments, the antibody consists of a single heavy chain sequence and a single light chain sequence or antigen-binding fragments thereof. In certain of the above described embodiments, the antibody is a chimeric antibody, a human antibody or a humanized antibody. In certain of the above described embodiments, the antibody is a monoclonal antibody.

### 4. BRIEF DESCRIPTION OF THE DRAWINGS

FIGS. 1A-1E. gRNA screening process and indel analysis for detecting knockout efficiencies. FIG. 1A shows a workflow to screen for potent gRNAs for each target. Three gRNAs targeting an early exon for each gene were designed using CRISPR Guide RNA Design software (Benchling), each gRNA was complexed with Cas9 protein and transfected into cells. Genomic DNA was isolated and the edited region was then PCR amplified, and the amplicon was Sanger sequenced. Sanger traces were analyzed using ICE software (Synthego) to determine editing efficiency. A wide range of indel efficiencies was observed for three gRNAs against gene A as depicted in FIG. 1B. Example of images demonstrating Synthego's ICE analysis for indel quantification for gene A gRNA-1 vs gRNA-3 as depicted in FIG. 1C. Confirmation of ICE results by Western blot. The level of protein production correlates with low and high efficiency gRNAs (as identified by ICE analysis of 9% and 65% indels) targeting the protein encoded by gene B. The image is representative of two biological replicates as depicted in FIG. 1D. Comparison of ICE results to TA cloning for three genes is shown in FIG. 1E (genes C, D, and E).

FIGS. 2A-2D. Optimization of the multiplex knockout method. An increasing amount of RNP targeting GFP was transfected into GFP expressing cells. Untransfected and Cas9-only transfected cells were used as controls as depicted in FIG. 2A. Different ratios of cr/tracrRNA were complexed to Cas9 protein targeting either gene F or gene G and indel percentages were measured. Mean and standard deviation of two biological replicates are shown in FIG. 2B. Comparison of different types of synthetic gRNA products (crRNA, XT-gRNA, and sgRNA) of the same sequence targeting the protein encoded by gene D, with untransfected CHO cells used as a control is depicted in FIG. 2C. Mean and standard deviation of two biological replicates are shown in FIG. 2C. Editing efficiency of six multiplexed gRNAs after three sequential transfections. Indel percentage was measured after each transfection is depicted in FIG. 2D.

FIGS. 3A-3C. CRISPR/Cas9 multiplex knockout method achieves high-efficiency knockouts confirmed by LC-MS/MS. A schematic displaying the multiplex gene editing approach. Individual gRNAs were first screened for each knockout target is shown in FIG. 3A. The most efficient gRNAs were multiplexed with Cas9 protein and transfected into cells sequentially to generate a highly (≥75% indel) edited pool of cells. Percent indel was measured at the pool stage of each target to obtain the probability of clones with all genes knocked out. After single cell cloning (SCC), clones were analyzed and screened through PCR and Sanger sequencing to identify those with all targets knocked out. Top clones were selected to initiate a fed-batch shake flask production cultures to characterize their growth profiles. At the end of the production culture, the harvested cell culture fluid (HCCF) was harvested and submitted for LC-MS/MS for verification of knockouts at the protein level. Percentage of indels for 10 multiplexed XT-gRNA targets after each of the four rounds of transfection is depicted in FIG. 3B. Comparison of KO efficiency for each gene in the 10× transfected pool (after the 4th sequential transfection); the predicted knockout efficiency of two alleles by squaring KO efficiency of the transfected pool for the respective gene; and the observed percentage of KO efficiency in single cell clones are depicted in FIG. 3C.

FIGS. 4A-4D. Growth characteristics of the 6× and 10× KO cell lines. Clones from the 6× KO cell line were screened and subjected to a fed-batch production assay to measure IVCC as depicted in FIG. 4A, and VCD as depicted in FIG. 4B. The parental CHO cell line was used as a wildtype control. Clones from the 10× KO cell line were screened and subjected to fed-batch production assays to measure IVCC as depicted in FIG. 4C, and VCD over culture duration as depicted in FIG. 4D. Mean and standard deviation of two biological replicates are shown.

### 5. DETAILED DESCRIPTION

The instant disclosure is directed to CRISPR/Cas9 knockout strategies and associated compositions as well as methods of utilizing cells modified by such knockout strategies to produce a product of interest, e.g., a recombinant protein.

The CRISPR/Cas9 knockout strategies described herein allow for drastically improved gene editing efficiencies. In certain embodiments, the CRISPR/Cas9 knockout strategies described herein allow for the simultaneous targeting of multiple genes in a single cell. In certain embodiments, the CRISPR/Cas9 knockout strategies described herein utilize RNP-based transfection of Cas9 protein. In certain embodiments, improved gene editing efficiencies are improved by employing specific RNP-to-cell ratios. In certain embodiments, improved gene editing efficiencies are improved by employing specific gRNA-to-Cas9 ratios. In certain embodiments, improved gene editing efficiencies are improved by employing different types of synthetic gRNAs.

In certain embodiments relating to the multiplex CRISPR/Cas9 knockout strategies described herein, high levels of gene interruption for all the targeted genes can be achieved at the pool stage, i.e., the point at which a portion of the population or “pool” of cells comprises edits in all targeted genes. Previous reports stated knockout efficiencies of 68% indel and >50% indel for a 3× KO pool (Grav et al., Biotechnology Journal. 2015;10(9):1446-1456) and a 10× KO (Amann et al., Deca CHO KO: exploring the limitations of CRISPR/Cas9 multiplexing in CHO cells. Design of Optimal CHO Protein N-glycosylation Profiles 2018:36) CHO cell pool, respectively. By comparison, the CRISPR/Cas9 knockout strategies described herein achieved >76% indel for the 6× KO and >84% indel for 10× KO CHO cell pools. Single cell cloning (SCC) of the respective pools allows for isolation of cell lines with all target genes knocked out. The CRISPR/Cas9 knockout strategies described herein significantly reduce the effort, time, and complexity of multiple gene knockout processes, and offer powerful tools for advancing host cell engineering.

For clarity, but not by way of limitation, the detailed description of the presently disclosed subject matter is divided into the following subsections:
5.1 Definitions;
5.2 CRISPR/Cas9 Knockout Strategies;
5.3 Cell Culture Methods; and
5.4 Products.

### 5.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one” and “one or more than one.”

The terms “comprise(s),” “include(s),” “having,” “has,” “can,” “contain(s)” and variants thereof, as used herein, are intended to be open-ended transitional phrases, terms or words that do not preclude the possibility of additional acts or structures. The present disclosure also contemplates other embodiments “comprising,” “consisting of” and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The terms “cell culture medium” and “culture medium” refer to a nutrient solution used for growing mammalian cells that typically provides at least one component from one or more of the following categories:
1) an energy source, usually in the form of a carbohydrate such as glucose;
2) all essential amino acids, and usually the basic set of twenty amino acids plus cysteine;
3) vitamins and/or other organic compounds required at low concentrations;
4) free fatty acids; and
5) trace elements, where trace elements are defined as inorganic compounds or naturally occurring elements that are typically required at very low concentrations, usually in the micromolar range.

The nutrient solution can optionally be supplemented with one or more components from any of the following categories:
1) hormones and other growth factors as, for example, insulin, transferrin, and epidermal growth factor;
2) salts and buffers as, for example, calcium, magnesium, and phosphate;
3) nucleosides and bases such as, for example, adenosine, thymidine, and hypoxanthine; and
4) protein and tissue hydrolysates

“Culturing” a cell refers to contacting a cell with a cell culture medium under conditions suitable to the survival and/or growth and/or proliferation of the cell.

“Batch culture” refers to a culture in which all components for cell culturing (including the cells and all culture nutrients) are supplied to the culturing bioreactor at the start of the culturing process.

“Fed-batch cell culture,” as used herein refers to a batch culture wherein the cells and culture medium are supplied to the culturing bioreactor initially, and additional culture nutrients are fed, continuously or in discrete increments, to the culture during the culturing process, with or without periodic cell and/or product harvest before termination of culture.

“Perfusion culture,” sometimes referred to as continuous culture, is a culture by which the cells are restrained in the culture by, e.g., filtration, encapsulation, anchoring to microcarriers, etc., and the culture medium is continuously, step-wise or intermittently introduced (or any combination of these) and removed from the culturing bioreactor.

As used herein, the term “cell,” refers to animal cells, mammalian cells, cultured cells, host cells, recombinant cells and recombinant host cells. Such cells are generally cell lines obtained or derived from mammalian tissues which are able to grow and survive when placed in media containing appropriate nutrients and/or growth factors.

The terms “host cell,” “host cell line” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny does not need to be completely identical in nucleic acid content to a parent cell, but can contain mutations. Mutant progeny that have the same function or biological activity as screened or selected for in the originally transformed cell are included herein.

The terms “mammalian cell” and “mammalian host cell” refers to cell lines derived from mammals. In certain embodiments, the cells are capable of growth and survival when placed in either monolayer culture or in suspension culture in a medium containing the appropriate nutrients and growth factors. The necessary growth factors for a particular cell line are readily determined empirically without undue experimentation, as described for example in Mammalian Cell Culture (Mather, J. P. ed., Plenum Press, N.Y. 1984), and Barnes and Sato, (1980) Cell, 22:649. In embodiments relating to the production of a product of interest, i.e., those relating to mammalian host cells, the cells are generally capable of expressing and secreting large quantities of a particular product, e.g., a protein of interest, into the culture medium. Examples of suitable mammalian host cells within the context of the present disclosure can include Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989); CHO-K1 (ATCC, CCL-61); monkey kidney CV1 line transformed by SV40 (COS-7, ATCC CRL 1651); human embryonic kidney line (293 or 293 cells subcloned for growth in suspension culture, Graham et al., J. Gen Virol., 36:59 1977); baby hamster kidney cells (BHK, ATCC CCL 10); mouse sertoli cells (TM4, Mather, Biol. Reprod., 23:243-251 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68 1982); MRC 5 cells; FS4 cells; and a human hepatoma line (Hep G2). In certain embodiments, the mammalian cells include Chinese hamster ovary cells/-DHFR (CHO, Urlaub and Chasin, Proc. Natl. Acad. Sci. USA, 77:4216 1980); dp12.CHO cells (EP 307,247 published 15 Mar. 1989).

In certain embodiments, the mammalian cells of the present disclosure include, but are not limited to “immunoresponsive cells.” Immunoresponsive cells refer to cells that function in an immune response, as well as progenitors or progeny thereof. In certain embodiments, the immunoresponsive cell is a cell of lymphoid lineage. Non-limiting examples of cells of lymphoid lineage include T-cells, Natural Killer (NK) cells, B cells, and stem cells from which lymphoid cells may be differentiated. In certain embodiments, the immunoresponsive cell is a cell of myeloid lineage. In certain embodiments, the immunoresponsive cell is an antigen presenting cell (“APC”). Non-limiting examples of APCs include macrophages, B cells, and dendritic cells.

The term “activity” as used herein with respect to activity of a protein refers to any activity of a protein including, but not limited to, enzymatic activity, ligand binding, drug transport, ion transport, protein localization, receptor binding, and/or structural activity. Such activity can be modulated, e.g., reduced or eliminated, by reducing or eliminating the expression of the protein, thereby reducing or eliminating the presence of the protein. Such activity can also be modulated, e.g., reduced or eliminated, by altering the nucleic acid sequence encoding the protein such that the resulting modified protein exhibits reduced or eliminated activity relative to a wild type protein.

The term “expression” or “expresses” are used herein to refer to transcription and translation occurring within a host cell. The level of expression of a product gene in a host cell can be determined on the basis of either the amount of corresponding mRNA that is present in the cell or the amount of the protein encoded by the product gene that is produced by the cell. For example, mRNA transcribed from a product gene is desirably quantitated by northern hybridization. Sambrook et al., Molecular Cloning: A Laboratory Manual, pp. 7.3-7.57 (Cold Spring Harbor Laboratory Press, 1989). Protein encoded by a product gene can be quantitated either by assaying for the biological activity of the protein or by employing assays that are independent of such activity, such as western blotting or radioimmunoassay using antibodies that are capable of reacting with the protein. Sambrook et al., Molecular Cloning: A Laboratory Manual, pp. 18.1-18.88 (Cold Spring Harbor Laboratory Press, 1989).

As used herein, “polypeptide” refers generally to peptides and proteins having more than about ten amino acids. The polypeptides can be homologous to the host cell, or preferably, can be exogenous, meaning that they are heterologous, i.e., foreign, to the host cell being utilized, such as a human protein produced by a Chinese hamster ovary cell, or a yeast polypeptide produced by a mammalian cell. In certain embodiments, mammalian polypeptides (polypeptides that were originally derived from a mammalian organism) are used, more preferably those which are directly secreted into the medium.

The term “protein” is meant to refer to a sequence of amino acids for which the chain length is sufficient to produce the higher levels of tertiary and/or quaternary structure. This is to distinguish from “peptides” or other small molecular weight drugs that do not have such structure. Typically, the protein herein will have a molecular weight of at least about 15-20 kD, preferably at least about 20 kD. Examples of proteins encompassed within the definition herein include host cell proteins as well as all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “antibody” is used herein in the broadest sense and encompasses various antibody structures including, but not limited to, monoclonal antibodies, polyclonal antibodies, monospecific antibodies (e.g., antibodies consisting of a single heavy chain sequence and a single light chain sequence, including multimers of such pairings), multispecific antibodies (e.g., bispecific antibodies) and antibody fragments so long as they exhibit the desired antigen-binding activity.

An “antibody fragment,” “antigen-binding portion” of an antibody (or simply “antibody portion”) or “antigen-binding fragment” of an antibody, as used herein, refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG and IgM, and several of these can be further divided into subclasses (isotypes), e.g., IgG₁, IgG₂, IgG₃, IgG₄, IgA₁, and IgA₂. In certain embodiments, the antibody is of the IgG₁ isotype. In certain embodiments, the antibody is of the IgG₂ isotype. The heavy chain constant domains that correspond to the different classes of immunoglobulins are called α, δ, ε, γ and μ, respectively. The light chain of an antibody can be assigned to one of two types, called kappa (κ) and lambda (λ), based on the amino acid sequence of its constant domain.

The term “titer” as used herein refers to the total amount of recombinantly expressed antibody produced by a cell culture divided by a given amount of medium volume. Titer is typically expressed in units of milligrams of antibody per milliliter or liter of medium (mg/ml or mg/L). In certain embodiments, titer is expressed in grams of antibody per liter of medium (g/L). Titer can be expressed or assessed in terms of a relative measurement, such as a percentage increase in titer as compared obtaining the protein product under different culture conditions.

The term “nucleic acid,” “nucleic acid molecule” or “polynucleotide” includes any compound and/or substance that comprises a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule can be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the disclosure in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see, e.g., Stadler et al, Nature Medicine 2017, published online 12 Jun. 2017, doi:10.1038/nm.4356 or EP 2 101 823 B1).

As used herein, the term “vector” refers to a nucleic acid molecule capable of transporting another nucleic acid to which it has been linked.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of anon-human antibody, and all or substantially all of the FRs correspond to those of a human antibody. A humanized antibody optionally can comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:

732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

An “immunoconjugate” is an antibody conjugated to one or more heterologous molecule(s), including but not limited to a cytotoxic agent.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the presently disclosed subject matter can be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain can be sufficient to confer antigen-binding specificity. Furthermore, antibodies that bind a particular antigen can be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

As used herein, the term “cell density” refers to the number of cells in a given volume of medium. In certain embodiments, a high cell density is desirable in that it can lead to higher protein productivity. Cell density can be monitored by any technique known in the art, including, but not limited to, extracting samples from a culture and analyzing the cells under a microscope, using a commercially available cell counting device or by using a commercially available suitable probe introduced into the bioreactor itself (or into a loop through which the medium and suspended cells are passed and then returned to the bioreactor).

As used herein, the term “recombinant cell” refers to cells which have some genetic modification from the original parent cells from which they are derived. Such genetic modification can be the result of an introduction of a heterologous gene for expression of the gene product, e.g., a recombinant protein.

As used herein, the term “recombinant protein” refers generally to peptides and proteins, including antibodies. Such recombinant proteins are “heterologous,” i.e., foreign to the host cell being utilized, such as an antibody produced by CHO cells.

### 5.2. CRISPR/Cas9 Knockout Strategies

In certain embodiments, the CRISPR/Cas9 knockout strategies described herein involve RNP-based transfection. Such RNP-based strategies can be more efficient than the plasmid-based delivery of Cas9 and gRNA and eliminates the possibility of plasmid Cas9 DNA integration into the CHO genome. Moreover, the lengthy and laborious cloning steps involved in plasmid-based delivery systems can be avoided by using the relatively quick and inexpensive synthesis of gRNA, which also allows for simultaneous testing of multiple gRNA sequences. Coupled with quantitative indel analysis of Sanger sequencing traces with Inference of CRISPR Edits (“ICE”) software, the strategies described herein enable swift identification of the most efficient gRNA sequence for each target gene. Notably, multiplexing many gRNAs into a single RNP transfection did not lower the efficiency of individual gRNAs. The ability to disrupt multiple genes simultaneously (e.g., 2, 3, 4, 5, 6, 7, 8, 9, 10, or more genes) reduces both the labor and time required to engineer knockout cells. Additionally, the modified cells generated using the strategies described herein have similar growth characteristics as the parental wildtype control. The strategies described herein can be adapted to engineer a wide variety of cells, including, but not limited to T cells, NK cells, B cells, macrophages, and dendritic cells, as well as any of a variety of mammalian host cells, e.g., CHO cells, COS-7 cells; HEK 293 cells, BHK cells, TM4 cells, CV1 cells; VERO-76 cells; HELA cells; and MDCK cells, having enhanced productivities and product attributes.

### 5.2.1. Identification of Efficient gRNAs

To identify a efficient gRNA for each target gene, transfections of purified Cas9 protein bound to candidate gRNAs in an RNP complex can be analyzed to individually or simultaneously screen several candidate gRNAs for a given locus. For quantification of editing efficiencies, the type and abundance of Cas9-induced edits can be determined. For example, but not by way of limitation, ICE, an online software for analyzing Sanger sequencing data, which has been extensively validated for targeted NGS, can be used to identify the type and quantitatively infer the abundance of Cas9 induced edits.

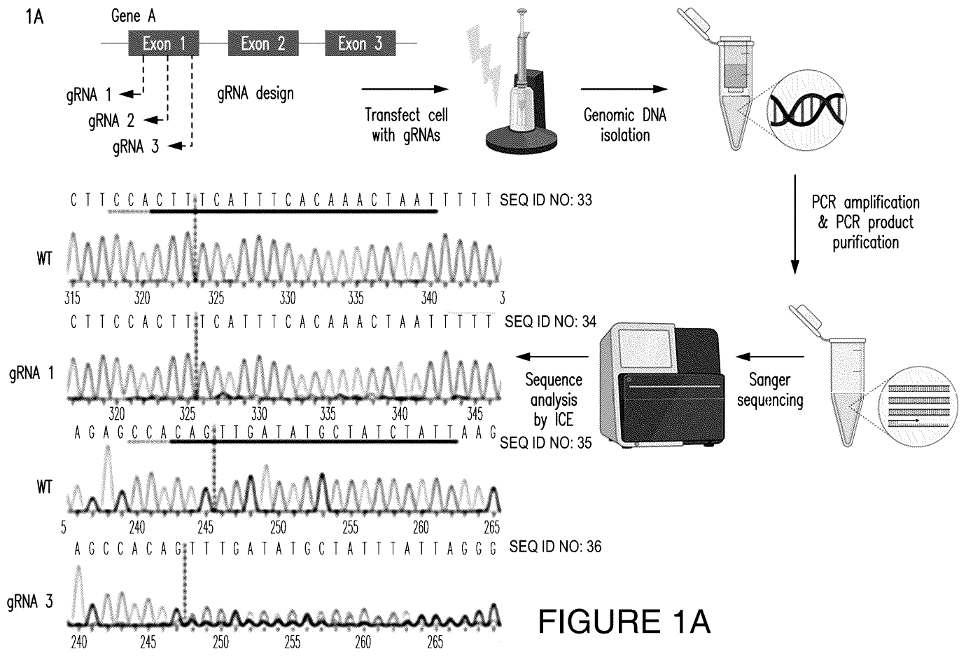

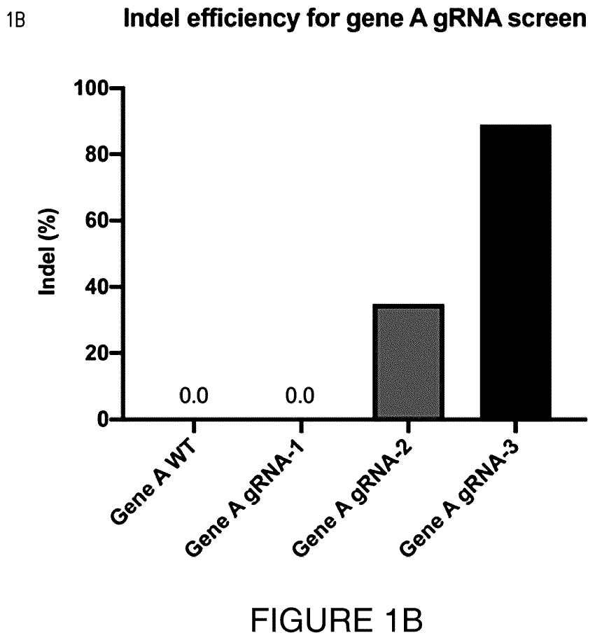

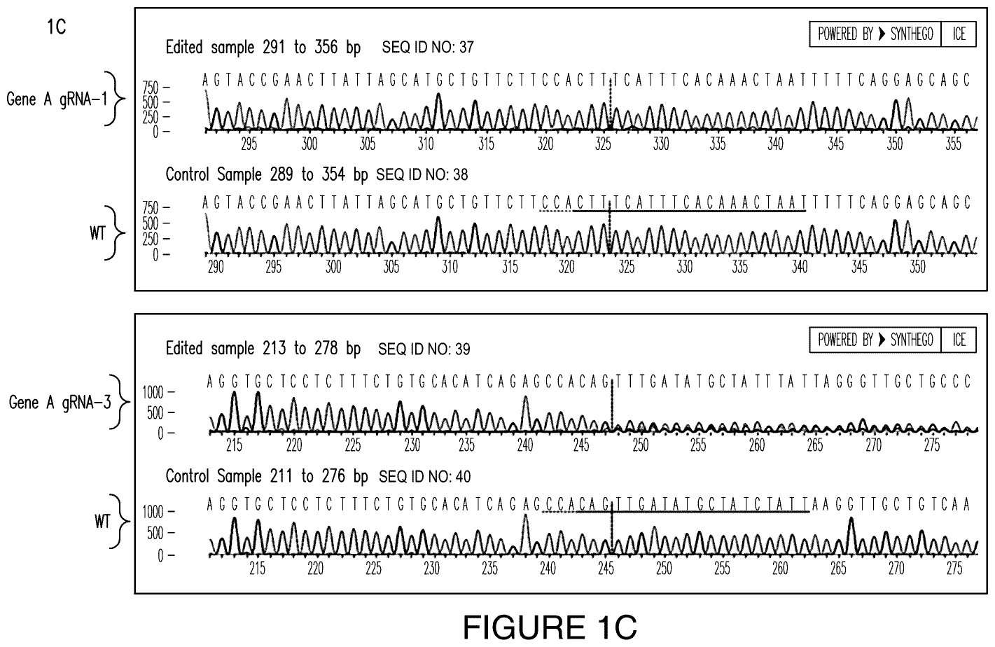

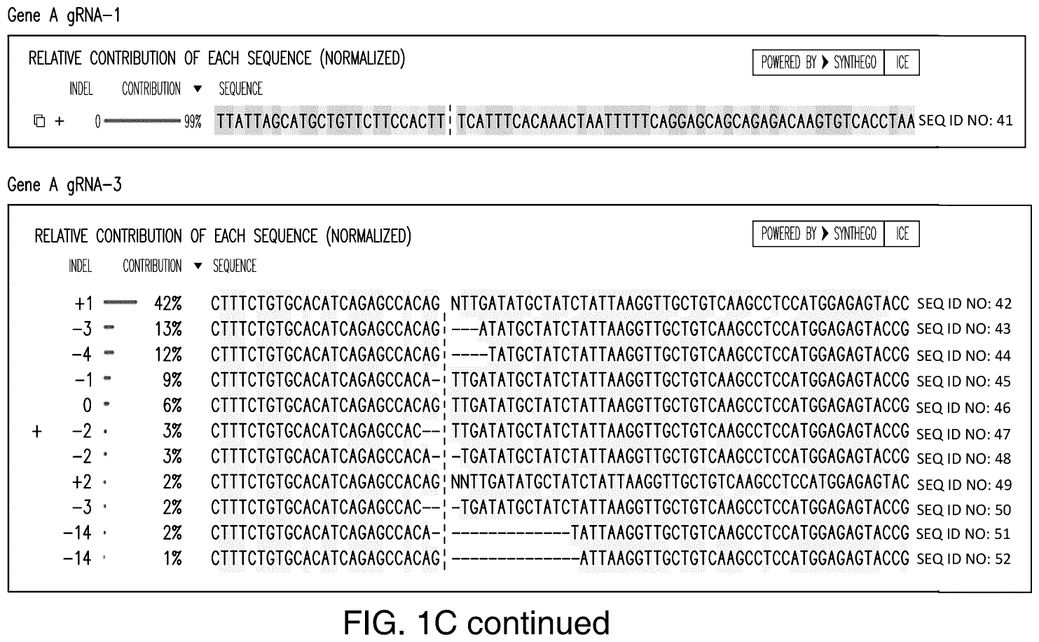

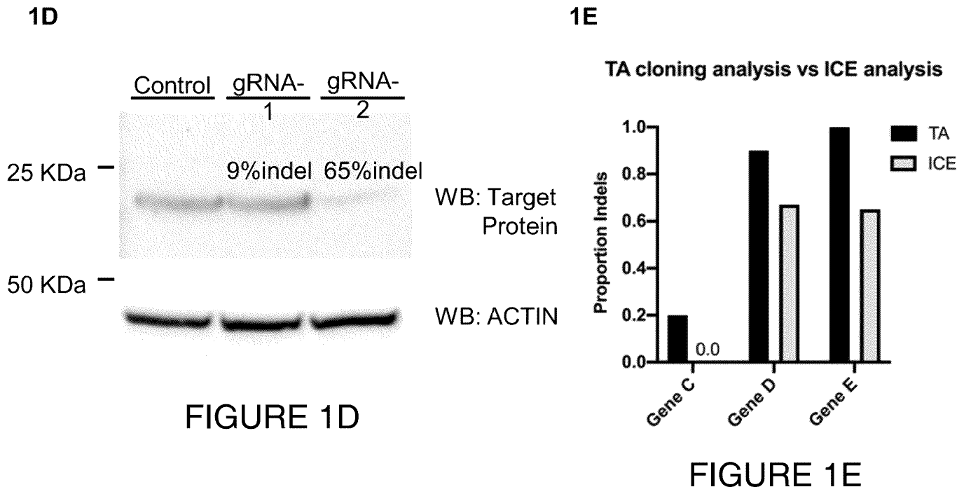

An exemplary workflow employing the strategies described herein (FIG. 1A) can accomplish transfection of cells with RNP, extraction of DNA from the transfected cells, amplification of the region surrounding the gRNA cut sites, and analysis of the sequenced amplicon. In certain embodiments, workflows employing the strategies described herein can be completed in about four days. In certain embodiments, workflows employing the strategies described herein allow for the rapid identification of efficient gRNAs from those with far lower editing efficiency.

In certain embodiments of the strategies described herein, the candidate gRNAs are sgRNAs. In certain embodiments of the strategies described herein, the candidate gRNAs comprise a crRNA and a tracrRNA. In certain embodiments of the strategies described herein, for example, if the gRNA is identified as having limited efficiency in directing CRISPR/Cas9-mediated indels, the crRNA is an XT-gRNA.

### 5.2.2. RNP Compositions & Transfection

In certain embodiments, the CRISPR/Cas9 knockout strategies described herein utilize RNP-based transfection of Cas9 protein. In certain embodiments, the strategies described herein utilize sequential rounds of transfection of one or more RNP compositions. In certain embodiments, the strategies described herein utilize RNP compositions comprising specific gRNA to Cas9 protein ratios. In certain embodiments, the strategies described herein utilize transfections comprising specific RNP to cell number ratios.

In certain embodiments, sequential rounds of transfection with gRNAs can generate a final pool of cells with higher levels of simultaneous knockout efficiency. For example, two or more gRNAs, including but not limited to gRNAs with varying levels of editing efficiency, can be mixed with Cas9 protein to form RNPs that are then employed to serially transfect cells, e.g., T cells, NK cells, B cells, dendritic cells, or CHO cells. In certain embodiments, cells, e.g., T cells, NK cells, B cells, dendritic cells, or CHO cells, can be transfected with the RNP two or more sequential times. In certain embodiments, additional RNPs, including RNPs comprising distinct gRNAs, can be transfected alone or in combination with the prior transfected RNPs in additional rounds of transfection. In certain embodiments, indel efficiency can be measured after each round of transfection by, for example, PCR and ICE analysis. In certain embodiments, the methods described herein involve serially transfecting a population of cells with the RNP until at least about 10% indel formation is achieved at each target locus. In certain embodiments, the methods described herein involve serially transfecting a population of cells with the RNP until at least about 15% indel formation is achieved at each target locus. In certain embodiments, the methods described herein involve serially transfecting a population of cells with the RNP until at least about 20% indel formation is achieved at each target locus. In certain embodiments, the methods described herein involve serially transfecting a population of cells with the RNP until at least about 25% indel formation is achieved at each target locus. In certain embodiments, the methods described herein involve serially transfecting a population of cells with the RNP until at least about 30% indel formation is achieved at each target locus. In certain embodiments, the methods described herein involve serially transfecting a population of cells with the RNP until at least about 35% indel formation is achieved at each target locus. In certain embodiments, the methods described herein involve serially transfecting a population of cells with the RNP until at least about 40% indel formation is achieved at each target locus. In certain embodiments, the methods described herein involve serially transfecting a population of cells with the RNP until at least about 45% indel formation is achieved at each target locus. In certain embodiments, the methods described herein involve serially transfecting a population of cells with the RNP until at least about 50% indel formation is achieved at each target locus. In certain embodiments, the methods described herein involve serially transfecting a population of cells with the RNP until at least about 55% indel formation is achieved at each target locus. In certain embodiments, the methods described herein involve serially transfecting a population of cells with the RNP until at least about 60% indel formation is achieved at each target locus. In certain embodiments, the methods described herein involve serially transfecting a population of cells with the RNP until at least about 70% indel formation is achieved at each target locus. In certain embodiments, the methods described herein involve serially transfecting a population of cells with the RNP until at least about 75% indel formation is achieved at each target locus. In certain embodiments, the methods described herein involve serially transfecting a population of cells with the RNP until at least about 80% indel formation is achieved at each target locus. In certain embodiments, the methods described herein involve serially transfecting a population of cells with the RNP until at least about 85% indel formation is achieved at each target locus. In certain embodiments, the methods described herein involve serially transfecting a population of cells with the RNP until at least about 90% indel formation is achieved at each target locus. In certain embodiments, the methods described herein involve serially transfecting a population of cells with the RNP until at least about 95% indel formation is achieved at each target locus.

In certain embodiments, gene editing efficiencies are improved by employing specific gRNA-to-Cas9 protein ratios during transfection. As outlined herein, the gRNAs can not only be present at specific ratios with respect to the Cas9 protein, but the gRNAs can be present in specific formats, e.g., sgRNA or hybrized crRNA/tracrRNA, and composition, e.g., conventional RNA and/or modified RNAs, such as XT-RNA. In certain embodiments, the ratio of gRNA to Cas9 protein is about 0.1 to about 1. In certain embodiments, the ratio of gRNA to Cas9 protein is about 0.2 to about 1. In certain embodiments, the ratio of gRNA to Cas9 protein is about 0.5 to about 1. In certain embodiments, the ratio of gRNA to Cas9 protein is about 0.75 to about 1. In certain embodiments, the ratio of gRNA to Cas9 protein is about 1 to about 1. In certain embodiments, the ratio of gRNA to Cas9 protein is about 2 to about 1. In certain embodiments, the ratio of gRNA to Cas9 protein is about 3 to about 1. In certain embodiments, the ratio of gRNA to Cas9 protein is about 4 to about 1. In certain embodiments, the ratio of gRNA to Cas9 protein is about 5 to about 1.

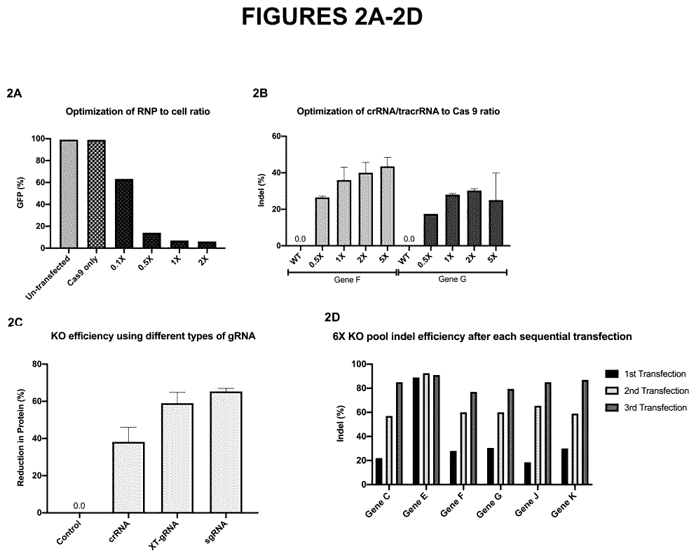

In certain embodiments, gene editing efficiencies are improved by employing specific RNP-to-cell ratios during transfection. In certain embodiments, the RNP-to-cell ratio is about 0.1 pmol to about 5 pmol RNP per million cells. In certain embodiments, the RNP-to-cell ratio is about 0.14 pmol RNP per million cells. In certain embodiments, the RNP-to-cell ratio is about 0.15 pmol RNP per million cells. In certain embodiments, the RNP-to-cell ratio is about 0.16 pmol RNP per million cells. In certain embodiments, the RNP-to-cell ratio is about 0.17 pmol RNP per million cells. In certain embodiments, the RNP-to-cell ratio is about 0.18 pmol RNP per million cells. In certain embodiments, the RNP-to-cell ratio is about 0.19 pmol RNP per million cells. In certain embodiments, the RNP-to-cell ratio is about 0.2 pmol RNP per million cells. In certain embodiments, the RNP-to-cell ratio is about 0.25 pmol RNP per million cells. In certain embodiments, the RNP-to-cell ratio is about 0.3 pmol RNP per million cells. In certain embodiments, the RNP-to-cell ratio is about 0.35 pmol RNP per million cells. In certain embodiments, the RNP-to-cell ratio is about 0.4 pmol RNP per million cells. In certain embodiments, the RNP-to-cell ratio is about 0.45 pmol RNP per million cells. In certain embodiments, the RNP-to-cell ratio is about 0.5 pmol RNP per million cells. In certain embodiments, the RNP-to-cell ratio is about 0.55 pmol RNP per million cells. In certain embodiments, the RNP-to-cell ratio is about 0.6 pmol RNP per million cells. In certain embodiments, the RNP-to-cell ratio is about 0.65 pmol RNP per million cells. In certain embodiments, the RNP-to-cell ratio is about 0.7 pmol RNP per million cells. In certain embodiments, the RNP-to-cell ratio is about 0.75 pmol RNP per million cells. In certain embodiments, the RNP-to-cell ratio is about 0.8 pmol RNP per million cells. In certain embodiments, the RNP-to-cell ratio is about 0.85 pmol RNP per million cells. In certain embodiments, the RNP-to-cell ratio is about 0.9 pmol RNP per million cells. In certain embodiments, the RNP-to-cell ratio is about 0.95 pmol RNP per million cells. In certain embodiments, the RNP-to-cell ratio is about 1 pmol RNP per million cells. In certain embodiments, the RNP-to-cell ratio is about 1.25 pmol RNP per million cells. In certain embodiments, the RNP-to-cell ratio is about 1.5 pmol RNP per million cells. In certain embodiments, the RNP-to-cell ratio is about 1.75 pmol RNP per million cells. In certain embodiments, the RNP-to-cell ratio is about 2 pmol RNP per million cells. In certain embodiments, the RNP-to-cell ratio is about 2.25 pmol RNP per million cells. In certain embodiments, the RNP-to-cell ratio is about 2.5 pmol RNP per million cells. In certain embodiments, the RNP-to-cell ratio is about 2.75 pmol RNP per million cells. In certain embodiments, the RNP-to-cell ratio is about 3 pmol RNP per million cells. In certain embodiments, the RNP-to-cell ratio is about 3.25 pmol RNP per million cells. In certain embodiments, the RNP-to-cell ratio is about 3.5 pmol RNP per million cells. In certain embodiments, the RNP-to-cell ratio is about 3.75 pmol RNP per million cells. In certain embodiments, the RNP-to-cell ratio is about 4 pmol RNP per million cells. In certain embodiments, the RNP-to-cell ratio is about 5 pmol RNP per million cells. For example, but not by way of limitation, about 0.7 pmol RNP to about 3.3 pmol RNP per million cells (0.1× to 2× concentrations in FIG. 2A) can be employed.

### 5.2.3. Multiplex RNP Transfections

In certain embodiments, the present disclosure relates to methods for modulating the expression of one or more cellular proteins by editing a gene encoding the cellular protein. In certain embodiments, the expression of one cellular protein is modulated by editing a gene encoding the cellular protein. In certain embodiments, the expression of two cellular proteins is modulated by editing genes encoding the cellular proteins. In certain embodiments, the expression of three cellular proteins is modulated by editing genes encoding the cellular proteins. In certain embodiments, the expression of four cellular proteins is modulated by editing genes encoding the cellular proteins. In certain embodiments, the expression of two cellular proteins is modulated by editing genes encoding the cellular proteins. In certain embodiments, the expression of three cellular proteins is modulated by editing genes encoding the cellular proteins. In certain embodiments, the expression of four cellular proteins is modulated by editing genes encoding the cellular proteins. In certain embodiments, the expression of five cellular proteins is modulated by editing genes encoding the cellular proteins. In certain embodiments, the expression of six cellular proteins is modulated by editing genes encoding the cellular proteins. In certain embodiments, the expression of seven cellular proteins is modulated by editing genes encoding the cellular proteins. In certain embodiments, the expression of eight cellular proteins is modulated by editing genes encoding the cellular proteins. In certain embodiments, the expression of nine cellular proteins is modulated by editing genes encoding the cellular proteins. In certain embodiments, the expression of ten cellular proteins is modulated by editing genes encoding the cellular proteins. In certain embodiments, the expression of eleven cellular proteins is modulated by editing genes encoding the cellular proteins. In certain embodiments, the expression of twelve cellular proteins is modulated by editing genes encoding the cellular proteins. In certain embodiments, the expression of thirteen cellular proteins is modulated by editing genes encoding the cellular proteins. In certain embodiments, the expression of fourteen cellular proteins is modulated by editing genes encoding the cellular proteins. In certain embodiments, the expression of fifteen or more cellular proteins is modulated by editing genes encoding the cellular proteins.

In certain embodiments, the present disclosure relates to methods for modulating the expression of one or more cellular proteins by editing a gene encoding the cellular protein. In certain embodiments, the expression of one cellular protein is modulated by editing a gene encoding the cellular protein. In certain embodiments, the expression of two cellular proteins is modulated by editing genes encoding the cellular proteins. In certain embodiments, the expression of three cellular proteins is modulated by editing genes encoding the cellular proteins. In certain embodiments, the expression of four cellular proteins is modulated by editing genes encoding the cellular proteins. In certain embodiments, the expression of two cellular proteins is modulated by editing genes encoding the cellular proteins. In certain embodiments, the expression of three cellular proteins is modulated by editing genes encoding the cellular proteins. In certain embodiments, the expression of four cellular proteins is modulated by editing genes encoding the cellular proteins. In certain embodiments, the expression of five cellular proteins is modulated by editing genes encoding the cellular proteins. In certain embodiments, the expression of six cellular proteins is modulated by editing genes encoding the cellular proteins. In certain embodiments, the expression of seven cellular proteins is modulated by editing genes encoding the cellular proteins. In certain embodiments, the expression of eight cellular proteins is modulated by editing genes encoding the cellular proteins. In certain embodiments, the expression of nine cellular proteins is modulated by editing genes encoding the cellular proteins. In certain embodiments, the expression of ten cellular proteins is modulated by editing genes encoding the cellular proteins. In certain embodiments, the expression of eleven cellular proteins is modulated by editing genes encoding the cellular proteins. In certain embodiments, the expression of twelve cellular proteins is modulated by editing genes encoding the cellular proteins. In certain embodiments, the expression of thirteen cellular proteins is modulated by editing genes encoding the cellular proteins. In certain embodiments, the expression of fourteen cellular proteins is modulated by editing genes encoding the cellular proteins. In certain embodiments, the expression of fifteen or more cellular proteins is modulated by editing genes encoding the cellular proteins.

In certain embodiments, one or more of the cellular proteins having expression modulated by the methods describe herein include, but are not limited to, proteins having enzymatic activity. In certain embodiments, one or more of the cellular proteins having expression modulated by the methods describe herein is a lipase, an esterase, or a hydrolase. For example, but not by way of limitation, methods for modulating enzyme activity, including but not limited to lipase, esterase, and/or hydrolase proteins, in a cellular include reducing or eliminating the expression of the corresponding polypeptide. In certain embodiments, a recombinant cellular is modified to reduce or eliminate the expression of one or more cellular protein relative to the expression of the protein in an unmodified cell.

In certain embodiments, the expression of a polypeptide in a cell that has been modified to reduce or eliminate the expression of the polypeptide is less than about 90%, less than about 80%, less than about 70%, less than about 60%, less than about 50%, less than about 40%, less than about 30%, less than about 20%, less than about 10%, less than about 5%, less than about 4%, less than about 3%, less than about 2% or less than about 1% of the corresponding polypeptide expression of a reference cell, e.g., an unmodified/wild type (WT) T cell, a WT NK cell, a WT B cell, a WT dendritic cell, or a WT CHO cell.

In certain embodiments, the expression of a polypeptide in a cell that has been modified to reduce or eliminate the expression of the polypeptide is at least about 90%, at least about 80%, at least about 70%, at least about 60%, at least about 50%, at least about 15 40%, at least about 30%, at least about 20%, at least about 10%, at least about 5%, at least about 4%, at least about 3%, at least about 2% or at least about 1% of the corresponding polypeptide expression of a reference cell, e.g., a WT T cell, a WT NK cell, a WT B cell, a WT dendritic cell, or a WT CHO cell.

In certain embodiments, the expression of a particular polypeptide in a cell that has been modified to reduce or eliminate the expression of the polypeptide is no more than about 90%, no more than about 80%, no more than about 70%, no more than about 60%, no more than about 50%, no more than about 40%, no more than about 30%, no more than about 20%, no more than about 10%, no more than about 5%, no more than about 4%, no more than about 3%, no more than about 2% or no more than about 1% of the corresponding polypeptide expression of a reference cell, e.g., a WT T cell, a WT NK cell, a WT B cell, a WT dendritic cell, or a WT CHO cell.

In certain embodiments, the expression of a polypeptide in a cell that has been modified to reduce or eliminate the expression of the polypeptide is between about 1% and about 90%, between about 10% and about 90%, between about 20% and about 90%, between about 25% and about 90%, between about 30% and about 90%, between about 40% and about 90%, between about 50% and about 90%, between about 60% and about 90%, between about 70% and about 90%, between about 80% and about 90%, between about 85% and about 90%, between about 1% and about 80%, between about 10% and about 80%, between about 20% and about 80%, between about 30% and about 80%, between about 40% and about 80%, between about 50% and about 80%, between about 60% and about 80%, between about 70% and about 80%, between about 75% and about 80%, between about 1% and about 70%, between about 10% and about 70%, between about 20% and about 70%, between about 30% and about 70%, between about 40% and about 70%, between about 50% and about 70%, between about 60% and about 70%, between about 65% and about 70%, between about 1% and about 60%, between about 10% and about 60%, between about 20% and about 60%, between about 30% and about 60%, between about 40% and about 60%, between about 50% and about 60%, between about 55% and about 60%, between about 1% and about 50%, between about 10% and about 50%, between about 20% and about 50%, between about 30% and about 50%, between about 40% and about 50%, between about 45% and about 50%, between about 1% and about 40%, between about 10% and about 40%, between about 20% and about 40%, between about 30% and about 40%, between about 35% and about 40%, between about 1% and about 30%, between about 10% and about 30%, between about 20% and about 30%, between about 25% and about 30%, between about 1% and about 20%, between about 5% and about 20%, between about 10% and about 20%, between about 15% and about 20%, between about 1% and about 10%, between about 5% and about 10%, between about 5% and about 20%, between about 5% and about 30%, between about 5% and about 40% of the corresponding polypeptide expression of a reference cell, e.g., a WT T cell, a WT NK cell, a WT B cell, a WT dendritic cell, or a WT CHO cell.

In certain embodiments, the expression of a polypeptide in a cell that has been modified to reduce or eliminate the expression of the polypeptide is between about 5% and about 40% of the corresponding polypeptide expression of a reference cell, e.g., a WT T cell, a WT NK cell, a WT B cell, a WT dendritic cell, or a WT CHO cell. In certain embodiments, the expression of a polypeptide in a cell that has been modified to reduce or eliminate the expression of the polypeptide is between about 5% and about 40% of the corresponding polypeptide expression of a reference cell, e.g., a WT T cell, a WT NK cell, a WT B cell, a WT dendritic cell, or a WT CHO cell. The expression of the polypeptide in different reference cells (e.g., cells that comprise at least one or both wild-type alleles of the corresponding gene) can vary.

### 5.3. Modified Cells

In certain embodiments, the cell modified in accordance with the instant disclosure is selected from the group consisting of cells of lymphoid lineage and cells of myeloid lineage. In certain embodiments, the cell is an immunoresponsive cell.

In certain embodiments, cells of the lymphoid lineage can provide production of antibodies, regulation of cellular immune system, detection of foreign agents in the blood, detection of cells foreign to the host, and the like. Non-limiting examples of cells of the lymphoid lineage include T-cells, NK cells, B cells, and stem cells from which lymphoid cells may be differentiated. In certain embodiments, the stem cell is a pluripotent stem cell (e.g., embryonic stem cell or an induced pluripotent stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells), Regulatory T cells (also known as suppressor T cells), tumor-infiltrating lymphocyte (TIL), Natural killer T cells, Mucosal associated invariant T cells, and γδ T cells. Cytotoxic T-cells (CTL or killer T cells) are a subset of T lymphocytes capable of inducing the death of infected somatic or tumor cells. In certain embodiments, the immunoresponsive cell is a T cell. The T cell can be a CD4⁺ T-cell or a CD8⁺ T cell. In certain embodiments, the T cell is a CD4⁺ T cell. In certain embodiments, the T cell is a CD8⁺ T cell. Non-limiting examples of the loci that can be edited in connection with the methods described herein include a TRAC locus, a TRBC locus, a TRDC locus, and a TRGC locus. In certain embodiments, the locus is a TRAC locus or a TRBC locus.

In certain embodiments, the cell is a NK cell. Natural killer (NK) cells can be lymphocytes that are part of cell-mediated immunity and act during the innate immune response.

In certain embodiments, the cells of the presently disclosed subject matter can be cells of the myeloid lineage. Non-limiting examples of cells of the myeloid lineage include monocytes, macrophages, neutrophils, dendritic cells, basophils, neutrophils, eosinophils, megakaryocytes, mast cell, erythrocyte, thrombocytes, and stem cells from which myeloid cells may be differentiated. In certain embodiments, the stem cell is a pluripotent stem cell (e.g., an embryonic stem cell or an induced pluripotent stem cell).

### 5.4. Cell Culture of Modified Cells

In certain embodiments, the present disclosure provides methods for producing a product, e.g., a polypeptide, of interest comprising culturing a modified cell disclosed herein. Suitable culture conditions for mammalian cells known in the art can be used for culturing the cells herein (J. Immunol. Methods (1983) 56:221-234) or can be easily determined by the skilled artisan (see, for example, Animal Cell Culture: A Practical Approach 2nd Ed., Rickwood, D. and Hames, B. D., eds. Oxford University Press, New York (1992)).

Mammalian cell culture can be prepared in a medium suitable for the particular cell being cultured. Commercially available media such as Ham's F10 (Sigma), Minimal Essential Medium (MEM, Sigma), RPMI-1640 (Sigma) and Dulbecco's Modified Eagle's Medium (DMEM, Sigma) are exemplary nutrient solutions. In addition, any of the media described in Ham and Wallace, (1979) Meth. Enz., 58:44; Barnes and Sato, (1980) Anal. Biochem., 102:255; U.S. Pat. Nos. 4,767,704; 4,657,866; 4,927,762; 5,122,469 or 4,560,655; International Publication Nos. WO 90/03430; and WO 87/00195; the disclosures of all of which are incorporated herein by reference, can be used as culture media. Any of these media can be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as gentamycin (gentamicin), trace elements (defined as inorganic compounds usually present at final concentrations in the micromolar range) lipids (such as linoleic or other fatty acids) and their suitable carriers, and glucose or an equivalent energy source. Any other necessary supplements can also be included at appropriate concentrations that would be known to those skilled in the art.

In certain embodiments, the mammalian cell that has been modified to reduce and/or eliminate the expression of a particular polypeptide is a CHO cell. Any suitable medium can be used to culture the CHO cell. In certain embodiments, a suitable medium for culturing the CHO cell can contain a basal medium component such as a DMEM/HAM F-12 based formulation (for composition of DMEM and HAM F12 media, see culture media formulations in American Type Culture Collection Catalogue of Cell Lines and Hybridomas, Sixth Edition, 1988, pages 346-349) (the formulation of medium as described in U.S. Pat. No. 5,122,469 are particularly appropriate) with modified concentrations of some components such as amino acids, salts, sugar, and vitamins, and optionally containing glycine, hypoxanthine, and thymidine; recombinant human insulin, hydrolyzed peptone, such as Primatone HS or Primatone RL (Sheffield, England), or the equivalent; a cell protective agent, such as Pluronic F68 or the equivalent pluronic polyol; gentamycin; and trace elements.

In certain embodiments, the mammalian cell that has been modified to reduce and/or eliminate the expression of a particular polypeptide is a cell that expresses a recombinant protein. The recombinant protein can be produced by growing cells which express the products of interest under a variety of cell culture conditions. For instance, cell culture procedures for the large or small-scale production of proteins are potentially useful within the context of the present disclosure. Procedures including, but not limited to, a fluidized bed bioreactor, hollow fiber bioreactor, roller bottle culture, shake flask culture, or stirred tank bioreactor system can be used, in the latter two systems, with or without microcarriers, and operated alternatively in a batch, fed-batch, or continuous mode.

In certain embodiments, the cell culture of the present disclosure is performed in a stirred tank bioreactor system and a fed batch culture procedure is employed. In the fed batch culture, the mammalian host cells and culture medium are supplied to a culturing vessel initially and additional culture nutrients are fed, continuously or in discrete increments, to the culture during culturing, with or without periodic cell and/or product harvest before termination of culture. The fed batch culture can include, for example, a semi-continuous fed batch culture, wherein periodically whole culture (including cells and medium) is removed and replaced by fresh medium. Fed batch culture is distinguished from simple batch culture in which all components for cell culturing (including the cells and all culture nutrients) are supplied to the culturing vessel at the start of the culturing process. Fed batch culture can be further distinguished from perfusion culturing insofar as the supernatant is not removed from the culturing vessel during the process (in perfusion culturing, the cells are restrained in the culture by, e.g., filtration, encapsulation, anchoring to microcarriers etc. and the culture medium is continuously or intermittently introduced and removed from the culturing vessel).

In certain embodiments, the cells of the culture can be propagated according to any scheme or routine that can be suitable for the specific host cell and the specific production plan contemplated. Therefore, the present disclosure contemplates a single step or multiple step culture procedure. In a single step culture, the host cells are inoculated into a culture environment and the processes of the instant disclosure are employed during a single production phase of the cell culture. Alternatively, a multi-stage culture is envisioned. In the multi-stage culture cells can be cultivated in a number of steps or phases. For instance, cells can be grown in a first step or growth phase culture wherein cells, possibly removed from storage, are inoculated into a medium suitable for promoting growth and high viability. The cells can be maintained in the growth phase for a suitable period of time by the addition of fresh medium to the host cell culture.

In certain embodiments, fed batch or continuous cell culture conditions are devised to enhance growth of the mammalian cells in the growth phase of the cell culture. In the growth phase cells are grown under conditions and for a period of time that is maximized for growth. Culture conditions, such as temperature, pH, dissolved oxygen (dO₂) and the like, are those used with the particular host and will be apparent to the ordinarily skilled artisan. Generally, the pH is adjusted to a level between about 6.5 and 7.5 using either an acid (e.g., CO₂) or a base (e.g., Na₂CO₃ or NaOH). A suitable temperature range for culturing mammalian cells such as CHO cells is between about 30° to 38° C. and a suitable dO₂ is between 5-90% of air saturation.

At a particular stage the cells can be used to inoculate a production phase or step of the cell culture. Alternatively, as described above the production phase or step can be continuous with the inoculation or growth phase or step.

In certain embodiments, the culturing methods described in the present disclosure can further include harvesting the product from the cell culture, e.g., from the production phase of the cell culture. In certain embodiments, the product produced by the cell culture methods of the present disclosure can be harvested from the third bioreactor, e.g., production bioreactor. For example, but not by way of limitation, the disclosed methods can include harvesting the product at the completion of the production phase of the cell culture. Alternatively or additionally, the product can be harvested prior to the completion of the production phase. In certain embodiments, the product can be harvested from the cell culture once a particular cell density has been achieved. For example, but not by way of limitation, the cell density can be from about 2.0×10⁷ cells/mL to about 5.0×10⁷ cells/mL prior to harvesting.

In certain embodiments, harvesting the product from the cell culture can include one or more of centrifugation, filtration, acoustic wave separation, flocculation and cell removal technologies.

In certain embodiments, the product of interest can be secreted from the host cells or can be a membrane-bound, cytosolic or nuclear protein. In certain embodiments, soluble forms of the polypeptide can be purified from the conditioned cell culture media and membrane-bound forms of the polypeptide can be purified by preparing a total membrane fraction from the expressing cells and extracting the membranes with a nonionic detergent such as TRITON® X-100 (EMD Biosciences, San Diego, Calif). In certain embodiments, cytosolic or nuclear proteins can be prepared by lysing the host cells (e.g., by mechanical force, sonication and/or detergent), removing the cell membrane fraction by centrifugation and retaining the supernatant.

### 5.5 Products of Interest Produced by Modified Cells

While in certain embodiments it is a modified cell, itself, that can be employed, e.g., in the context of a cell-based therapy, in certain embodiments, cells modified as outlined herein can be employed to produce a product. The modified cells and/or methods of the present disclosure can thus be used to produce any product of interest that can be expressed by the cells disclosed herein.

In certain embodiments, the cells and/or methods of the present disclosure can be used for the production of polypeptides, e.g., mammalian polypeptides. Non-limiting examples of such polypeptides include hormones, receptors, fusion proteins, regulatory factors, growth factors, complement system factors, enzymes, clotting factors, anti-clotting factors, kinases, cytokines, CD proteins, interleukins, therapeutic proteins, diagnostic proteins and antibodies. The cells and/or methods of the present disclosure are not specific to the molecule, e.g., antibody, that is being produced.

In certain embodiments, the methods of the present disclosure can be used for the production of antibodies, including therapeutic and diagnostic antibodies or antigen-binding fragments thereof. In certain embodiments, the antibody produced by cell and methods of the present disclosure can be, but are not limited to, monospecific antibodies (e.g., antibodies consisting of a single heavy chain sequence and a single light chain sequence, including multimers of such pairings), multispecific antibodies and antigen-binding fragments thereof. For example, but not by way of limitation, the multispecific antibody can be a bispecific antibody, a biepitopic antibody, a T-cell-dependent bispecific antibody (TDB), a Dual Acting FAb (DAF) or antigen-binding fragments thereof.

### 5.5.1 Multispecific Antibodies

In certain aspects, an antibody produced by cells and methods provided herein is a multispecific antibody, e.g., a bispecific antibody. “Multispecific antibodies” are monoclonal antibodies that have binding specificities for at least two different sites, i.e., different epitopes on different antigens (i.e., bispecific) or different epitopes on the same antigen (i.e., biepitopic). In certain aspects, the multispecific antibody has three or more binding specificities. Multispecific antibodies can be prepared as full length antibodies or antibody fragments as described herein.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multispecific antibodies can also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tuft et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other non-limiting examples of multispecific antibodies with three or more antigen binding sites can be found in WO 2010/115589, WO 2010/112193, WO 2010/136172, WO 2010/145792 and WO 2013/026831. The bispecific antibody or antigen binding fragment thereof also includes a “Dual Acting FAb” or “DAF” (see, e.g., US 2008/0069820 and WO 2015/095539).

Multispecific antibodies can also be provided in an asymmetric form with a domain crossover in one or more binding arms of the same antigen specificity, i.e., by exchanging the VH/VL domains (see, e.g., WO 2009/080252 and WO 2015/150447), the CH1/CL domains (see, e.g., WO 2009/080253) or the complete Fab arms (see, e.g., WO 2009/080251, WO 2016/016299, also see Schaefer et al, PNAS, 108 (2011) 1187-1191, and Klein at al., MAbs 8 (2016) 1010-20). In certain embodiments, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See, e.g., WO 2016/172485.

Various further molecular formats for multispecific antibodies are known in the art and are included herein (see, e.g., Spiess et al., Mol. Immunol. 67 (2015) 95-106).

In certain embodiments, particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells.

Additional non-limiting examples of bispecific antibody formats that can be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., Prot. Eng. 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat. Rev. 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### 5.5.2 Antibody Fragments

In certain aspects, an antibody produced by the cells and methods provided herein is an antibody fragment. For example, but not by way of limitation, the antibody fragment is a Fab, Fab′, Fab′-SH or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In certain embodiments, the antibody fragment is a diabody, a triabody or a tetrabody. “Diabodies” are antibody fragments with two antigen-binding sites that can be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Plückthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody.

### 5.5.3 Chimeric and Humanized Antibodies

In certain aspects, an antibody produced by the cells and methods provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In certain embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5, 821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that can be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 5.5.4 Human Antibodies

In certain aspects, an antibody produced by the cells and methods provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals can be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

### 5.5.5 Target Molecules

Non-limiting examples of molecules that can be targeted by an antibody produced by the cells and methods disclosed herein include soluble serum proteins and their receptors and other membrane bound proteins (e.g., adhesins). In certain embodiments, an antibody produced by the cells and methods disclosed herein is capable of binding to one, two or more cytokines, cytokine-related proteins, and cytokine receptors selected from the group consisting of 8MPI, 8MP2, 8MP38 (GDFIO), 8MP4, 8MP6, 8MP8, CSFI (M-CSF), CSF2 (GM-CSF), CSF3 (G-CSF), EPO, FGF1 (αFGF), FGF2 (βFGF), FGF3 (int-2), FGF4 (HST), FGF5, FGF6 (HST-2), FGF7 (KGF), FGF9, FGF10, FGF11, FGF12, FGF12B, FGF14, FGF16, FGF17, FGF19, FGF20, FGF21, FGF23, IGF1, IGF2, IFNA1, IFNA2, IFNA4, IFNA5, IFNA6, IFNA7, IFN81, IFNG, IFNWI, FEL1, FEL1 (EPSELON), FEL1 (ZETA), IL 1A, IL 1B, IL2, IL3, IL4, IL5, IL6, IL7, IL8, IL9, IL10, IL 11, IL 12A, IL 12B, IL 13, IL 14, IL 15, IL 16, IL 17, IL 17B, IL 18, IL 19, IL20, IL22, IL23, IL24, IL25, IL26, IL27, IL28A, IL28B, IL29, IL30, PDGFA, PDGFB, TGFA, TGFB1, TGFB2, TGFBb3, LTA (TNF-β), LTB, TNF (TNF-α), TNFSF4 (OX40 ligand), TNFSF5 (CD40 ligand), TNFSF6 (FasL), TNFSF7 (CD27 ligand), TNFSF8 (CD30 ligand), TNFSF9 (4-1 BB ligand), TNFSF10 (TRAIL), TNFSF11 (TRANCE), TNFSF12 (APO3L), TNFSF13 (April), TNFSF13B, TNFSF14 (HVEM-L), TNFSF15 (VEGI), TNFSF18, HGF (VEGFD), VEGF, VEGFB, VEGFC, IL1R1, IL1R2, IL1RL1, IL1RL2, IL2RA, IL2RB, IL2RG, IL3RA, IL4R, IL5RA, IL6R, IL7R, IL8RA, IL8RB, IL9R, IL10RA, IL10RB, IL 11RA, IL12RB1, IL12RB2, IL13RA1, IL13RA2, IL15RA, IL17R, IL18R1, IL20RA, IL21R, IL22R, IL1HY1, IL1RAP, IL1RAPL1, IL1RAPL2, IL1RN, IL6ST, IL18BP, IL18RAP, IL22RA2, AIF1, HGF, LEP (leptin), PTN, and THPO.k

In certain embodiments, an antibody produced by cells and methods disclosed herein is capable of binding to a chemokine, chemokine receptor, or a chemokine-related protein selected from the group consisting of CCLI (1-309), CCL2 (MCP-1/MCAF), CCL3 (MIP-Iα), CCL4 (MIP-Iβ), CCLS (RANTES), CCL7 (MCP-3), CCL8 (mcp-2), CCL11 (eotaxin), CCL 13 (MCP-4), CCL 15 (MIP-Iδ), CCL 16 (HCC-4), CCL 17 (TARC), CCL 18 (PARC), CCL 19 (MDP-3b), CCL20 (MIP-3α), CCL21 (SLC/exodus-2), CCL22 (MDC/STC-1), CCL23 (MPIF-1), CCL24 (MPIF-2 /eotaxin-2), CCL25 (TECK), CCL26 (eotaxin-3), CCL27 (CTACK/ILC), CCL28, CXCLI (GROI), CXCL2 (GR02), CXCL3 (GR03), CXCL5 (ENA-78), CXCL6 (GCP-2), CXCL9 (MIG), CXCL 10 (IP 10), CXCL 11 (1-TAC), CXCL 12 (SDFI), CXCL 13, CXCL 14, CXCL 16, PF4 (CXCL4), PPBP (CXCL7), CX3CL 1 (SCYDI), SCYEI, XCLI (lymphotactin), XCL2 (SCM-Iβ), BLRI (MDR15), CCBP2 (D6/JAB61), CCRI (CKRI/HM145), CCR2 (mcp-IRB IRA), CCR3 (CKR3/CMKBR3), CCR4, CCR5 (CMKBR5/ChemR13), CCR6 (CMKBR6/CKR-L3/STRL22/DRY6), CCR7 (CKR7/EBII), CCR8 (CMKBR8/ TER1/CKR-L1), CCR9 (GPR-9-6), CCRL1 (VSHK1), CCRL2 (L-CCR), XCR1 (GPR5/CCXCR1), CMKLR1, CMKOR1 (RDC1), CX3CR1 (V28), CXCR4, GPR2 (CCR10), GPR31, GPR81 (FKSG80), CXCR3 (GPR9/CKR-L2), CXCR6 (TYMSTR/STRL33/Bonzo), HM74, IL8RA (IL8Rα), IL8RB (IL8Rβ), LTB4R (GPR16), TCP10, CKLFSF2, CKLFSF3, CKLFSF4, CKLFSF5, CKLFSF6, CKLFSF7, CKLFSF8, BDNF, C5, C5R1, CSF3, GRCC10 (C10), EPO, FY (DARC), GDF5, HDF1, HDF1α, DL8, PRL, RGS3, RGS13, SDF2, SLIT2, TLR2, TLR4, TREM1, TREM2, and VHL.

In certain embodiments, an antibody produced by methods disclosed herein (e.g., a multispecific antibody such as a bispecific antibody) is capable of binding to one or more target molecules selected from the following: 0772P (CA125, MUC16) (i.e., ovarian cancer antigen), ABCF1; ACVR1; ACVR1B; ACVR2; ACVR2B; ACVRL1; ADORA2A; Aggrecan; AGR2; AICDA; AIF1; AIG1; AKAP1; AKAP2; AMH; AMHR2; amyloid beta; ANGPTL; ANGPT2; ANGPTL3; ANGPTL4; ANPEP; APC; APOC1; AR; ASLG659; ASPHD1 (aspartate beta-hydroxylase domain containing 1; LOC253982); AZGP1 (zinc-a-glycoprotein); B7.1; B7.2; BAD; BAFF-R (B cell -activating factor receptor, BLyS receptor 3, BR3; BAG1; BAI1; BCL2; BCL6; BDNF; BLNK; BLRI (MDR15); BMP1; BMP2; BMP3B (GDF10); BMP4; BMP6; BMP8; BMPR1A; BMPR1B (bone morphogenic protein receptor-type IB); BMPR2; BPAG1 (plectin); BRCA1; Brevican; C19orf10 (IL27w); C3; C4A; C5; C5R1; CANT1; CASP1; CASP4; CAV1; CCBP2 (D6/JAB61); CCL1 (1-309); CCL11 (eotaxin); CCL13 (MCP-4); CCL15 (MIP1δ); CCL16 (HCC-4); CCL17 (TARC); CCL18 (PARC); CCL19 (MIP-3β); CCL2 (MCP-1); MCAF; CCL20 (MIP-3α); CCL21 (MTP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MTP-Iα); CCL4 (MDP-Iβ); CCLS(RANTES); CCL7 (MCP-3); CCL8 (mcp-2); CCNA1; CCNA2; CCND1; CCNE1; CCNE2; CCR1 (CKRI/HM145); CCR2 (mcp-IRβ/RA);CCR3 (CKR/ CMKBR3); CCR4; CCR5 (CMKBR5/ChemR13); CCR6 (CMKBR6/CKR-L3/STRL22/DRY6); CCR7 (CKBR7/EBI1); CCR8 (CMKBR8/TER1/CKR-L1); CCR9 (GPR-9-6); CCRL1 (VSHK1); CCRL2 (L-CCR); CD164; CD19; CD1C; CD20; CD200; CD22 (B-cell receptor CD22-B isoform); CD24; CD28; CD3; CD37; CD38; CD3E; CD3G; CD3Z; CD4; CD40; CD40L; CD44; CD45RB; CD52; CD69; CD72; CD74; CD79A (CD79α, immunoglobulin-associated alpha, a B cell-specific protein); CD79B; CDS; CD80; CD81; CD83; CD86; CDH1 (E-cadherin); CDH10; CDH12; CDH13; CDH18; CDH19; CDH20; CDH5; CDH7; CDH8; CDH9; CDK2; CDK3; CDK4; CDK5; CDK6; CDK7; CDK9; CDKN1A (p21/WAF1/Cip1); CDKN1B (p27/Kip1); CDKN1C; CDKN2A (P16INK4a); CDKN2B; CDKN2C; CDKN3; CEBPB; CER1; CHGA; CHGB; Chitinase; CHST10; CKLFSF2; CKLFSF3; CKLFSF4; CKLFSF5; CKLFSF6; CKLFSF7; CKLFSF8; CLDN3;CLDN7 (claudin-7); CLL-1 (CLEC12A, MICL, and DCAL2); CLN3; CLU (clusterin); CMKLR1; CMKOR1 (RDC1); CNR1; COL 18A1; COL1A1; COL4A3; COL6A1; complement factor D; CR2; CRP; CRIPTO (CR, CR1, CRGF, CRIPTO, TDGF1, teratocarcinoma-derived growth factor); CSFI (M-CSF); CSF2 (GM-CSF); CSF3 (GCSF); CTLA4; CTNNB1 (b-catenin); CTSB (cathepsin B); CX3CL1 (SCYDI); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCLS (ENA-78/LIX); CXCL6 (GCP-2); CXCL9 (MIG); CXCR3 (GPR9/CKR-L2); CXCR4; CXCR5 (Burkitt's lymphoma receptor 1, a G protein-coupled receptor); CXCR6 (TYMSTR/STRL33/Bonzo); CYB5; CYC1; CYSLTR1; DAB2IP; DES; DKFZp451J0118; DNCLI; DPP4; E16 (LAT1, SLC7A5); E2F1; ECGF1; EDG1; EFNA1; EFNA3; EFNB2; EGF; EGFR; ELAC2; ENG; ENO1; ENO2; ENO3; EPHB4; EphB2R; EPO; ERBB2 (Her-2); EREG; ERK8; ESR1; ESR2; ETBR (Endothelin type B receptor); F3 (TF); FADD; FasL; FASN; FCER1A; FCER2; FCGR3A; FcRH1 (Fc receptor-like protein 1); FcRH2 (IFGP4, IRTA4, SPAP1A (SH2 domain containing phosphatase anchor protein 1a), SPAP1B, SPAP1C); FGF; FGF1 (αFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGFS; FGF6 (HST-2); FGF7 (KGF); FGF8; FGF9; FGFR; FGFR3; FIGF (VEGFD); FEL1 (EPSILON); FIL1 (ZETA); FLJ12584; FLJ25530; FLRTI (fibronectin); FLT1; FOS; FOSL1 (FRA-1); FY (DARC); GABRP (GABAa); GAGEB1; GAGEC1; GALNAC4S-6ST; GATA3; GDFS; GDNF-Ra1 (GDNF family receptor alpha 1; GFRA1; GDNFR; GDNFRA; RETL1; TRNR1; RET1L; GDNFR-alpha1; GFR-ALPHA-1); GEDA; GFI1; GGT1; GM-CSF; GNASI; GNRHI; GPR2 (CCR10); GPR19 (G protein-coupled receptor 19; Mm.4787); GPR31; GPR44; GPR54 (KISS1 receptor; KISS1R; GPR54; HOT7T175; AXOR12); GPR81 (FKSG80); GPR172A (G protein-coupled receptor 172A; GPCR41; FLJ11856; D15Ertd747e);GRCCIO (C10); GRP; GSN (Gelsolin); GSTP1; HAVCR2; HDAC4; HDAC5; HDAC7A; HDAC9; HGF; HIF1A; HOP1; histamine and histamine receptors; HLA-A; HLA-DOB (Beta subunit of MHC class II molecule (Ia antigen); HLA-DRA; HM74; HMOXI ; HUMCYT2A; ICEBERG; ICOSL; 1D2; IFN-a; IFNA1; IFNA2; IFNA4; IFNA5; IFNA6; IFNA7; IFNB1; IFNgamma; DFNW1; IGBP 1; IGF1; IGF1R; IGF2; IGFBP2; IGFBP3; IGFBP6; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; ILIF10; IL1F5; IL1F6; IL1F7; IL1F8; IL1F9; IL1HY1; IL1R1; IL1R2; IL1RAP; IL1RAPL1; IL1RAPL2; IL1RL1; IL1RL2, ILIRN; IL2; IL20; IL20Rα; IL21 R; IL22; IL-22c; IL22R; IL22RA2; IL23; IL24; IL25; IL26; IL27; IL28A; IL28B; IL29; IL2RA; IL2RB; IL2RG; IL3; IL30; IL3RA; IL4; IL4R; IL5; IL5RA; IL6; IL6R; IL6ST (glycoprotein 130); influenza A; influenza B; EL7; EL7R; EL8; IL8RA; DL8RB; IL8RB; DL9; DL9R; DLK; INHA; INHBA; INSL3; INSL4; IRAK1; IRTA2 (Immunoglobulin superfamily receptor translocation associated 2); ERAK2; ITGA1; ITGA2; ITGA3; ITGA6 (a6 integrin); ITGAV; ITGB3; ITGB4 (b4 integrin); α4β7 and αEβ7 integrin heterodimers; JAG1; JAK1; JAK3; JUN; K6HF; KAI1; KDR; KITLG; KLF5 (GC Box BP); KLF6; KLKIO; KLK12; KLK13; KLK14; KLK15; KLK3; KLK4; KLK5; KLK6; KLK9; KRT1; KRT19 (Keratin 19); KRT2A; KHTHB6 (hair-specific type H keratin); LAMAS; LEP (leptin); LGR5 (leucine-rich repeat-containing G protein-coupled receptor 5; GPR49, GPR67); Lingo-p75; Lingo-Troy; LPS; LTA (TNF-b); LTB; LTB4R (GPR16); LTB4R2; LTBR; LY64 (Lymphocyte antigen 64 (RP105), type I membrane protein of the leucine rich repeat (LRR) family); Ly6E (lymphocyte antigen 6 complex, locus E; Ly67,RIG-E,SCA-2,TSA-1); Ly6G6D (lymphocyte antigen 6 complex, locus G6D; Ly6-D, MEGT1); LY6K (lymphocyte antigen 6 complex, locus K; LY6K; HSJ001348; FLJ35226); MACMARCKS; MAG or OMgp; MAP2K7 (c-Jun); MDK; MDP; MIB1; midkine; MEF; MIP-2; MKI67; (Ki-67); MMP2; MMP9; MPF (MPF, MSLN, SMR, megakaryocyte potentiating factor, mesothelin); MS4A1; MSG783 (RNF124, hypothetical protein FLJ20315);MSMB; MT3 (metallothionectin-111); MTSS1; MUC1 (mucin); MYC; MY088; Napi3b (also known as NaPi2b) (NAPI-3B, NPTIIb, SLC34A2, solute carrier family 34 (sodium phosphate), member 2, type II sodium-dependent phosphate transporter 3b); NCA; NCK2; neurocan; NFKB1; NFKB2; NGFB (NGF); NGFR; NgR-Lingo; NgR-Nogo66 (Nogo); NgR-p75; NgR-Troy; NME1 (NM23A); NOX5; NPPB; NR0B1; NR0B2; NR1D1; NR1D2; NR1H2; NR1H3; NR1H4; NR112; NR113; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT5E; NTN4; ODZI; OPRD1; OX40; P2RX7; P2X5 (Purinergic receptor P2X ligand-gated ion channel 5); PAP; PART1; PATE; PAWR; PCA3; PCNA; PD-L1; PD-L2; PD-1; POGFA; POGFB; PECAM1; PF4 (CXCL4); PGF; PGR; phosphacan; PIAS2; PIK3CG; PLAU (uPA); PLG; PLXDC1; PMEL17 (silver homolog; SILV; D12S53E; PMEL17; SI; SIL); PPBP (CXCL7); PPID; PRI; PRKCQ; PRKDI; PRL; PROC; PROK2; PSAP; PSCA hlg (2700050C12Rik, C530008O16Rik, RIKEN cDNA 2700050C12, RIKEN cDNA 2700050C12 gene); PTAFR; PTEN; PTGS2 (COX-2); PTN; RAC2 (p21 Rac2); RARB; RET (ret proto-oncogene; MEN2A; HSCR1; MEN2B; MTC1; PTC; CDHF12; Hs.168114; RET51; RET-ELE1); RGSI; RGS13; RGS3; RNF110 (ZNF144); ROBO2; S100A2; SCGB1D2 (lipophilin B); SCGB2A1 (mammaglobin2); SCGB2A2 (mammaglobin 1); SCYEI (endothelial Monocyte-activating cytokine); SDF2; Sema 5b (F1110372, KIAA1445, Mm.42015, SEMASB, SEMAG, Semaphorin 5b Hlog, sema domain, seven thrombospondin repeats (type 1 and type 1-like), transmembrane domain (TM) and short cytoplasmic domain, (semaphorin) 5B); SERPINA1; SERPINA3; SERP1NB5 (maspin); SERPINE1(PAI-1); SERPDMF1; SHBG; SLA2; SLC2A2; SLC33A1; SLC43A1; SLIT2; SPPI; SPRR1B (Sprl); ST6GAL1; STABI; STAT6; STEAP (six transmembrane epithelial antigen of prostate); STEAP2 (HGNC_8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein); TB4R2; TBX21; TCPIO; TOGFI; TEK; TENB2 (putative transmembrane proteoglycan); TGFA; TGFBI; TGFB1II; TGFB2; TGFB3; TGFBI; TGFBRI; TGFBR2; TGFBR3; THIL; THBSI (thrombospondin-1); THBS2; THBS4; THPO; TIE (Tie-1); TMP3; tissue factor; TLR1; TLR2; TLR3; TLR4; TLR5; TLR6; TLR7; TLR8; TLR9; TLR10; TMEFF1 (transmembrane protein with EGF-like and two follistatin-like domains 1; Tomoregulin-1); TMEM46 (shisa homolog 2); TNF; TNF-a; TNFAEP2 (B94); TNFAIP3; TNFRSFIIA; TNFRSF1A; TNFRSF1B; TNFRSF21; TNFRSF5; TNFRSF6 (Fas); TNFRSF7; TNFRSF8; TNFRSF9; TNFSF10 (TRAIL); TNFSF11 (TRANCE); TNFSF12 (AP03L); TNFSF13 (April); TNFSF13B; TNFSF14 (HVEM-L); TNFSF15 (VEGI); TNFSF18; TNFSF4 (OX40 ligand); TNFSFS (CD40 ligand); TNFSF6 (FasL); TNFSF7 (CD27 ligand); TNFSFS (CD30 ligand); TNFSF9 (4-1 BB ligand); TOLLIP; Toll-like receptors; TOP2A (topoisomerase Ea); TP53; TPM1; TPM2; TRADD; TMEM118 (ring finger protein, transmembrane 2; RNFT2; FLJ14627); TRAF1; TRAF2; TRAF3; TRAF4; TRAF5; TRAF6; TREM1; TREM2; TrpM4 (BR22450, F1120041, TRPM4, TRPM4B, transient receptor potential cation channel, subfamily M, member 4); TRPC6; TSLP; TWEAK; Tyrosinase (TYR; OCAIA; OCA1A; tyrosinase; SHEP3);VEGF; VEGFB; VEGFC; versican; VHL C5; VLA-4; XCL1 (lymphotactin); XCL2 (SCM-1b); XCRI(GPR5/CCXCRI); YY1; and ZFPM2.

In certain embodiments, an antibody produced by the cells and methods disclosed herein is capable of binding to CD proteins such as CD3, CD4, CDS, CD16, CD19, CD20, CD21 (CR2 (Complement receptor 2) or C3DR (C3d/Epstein Barr virus receptor) or Hs.73792); CD33; CD34; CD64; CD72 (B-cell differentiation antigen CD72, Lyb-2); CD79b (CD79B, CD79β, IGb (immunoglobulin-associated beta), B29); CD200 members of the ErbB receptor family such as the EGF receptor, HER2, HER3, or HER4 receptor; cell adhesion molecules such as LFA-1, Mac1, p150.95, VLA-4, ICAM-1, VCAM, alpha4/beta7 integrin, and alphav/beta3 integrin including either alpha or beta subunits thereof (e.g., anti-CD11a, anti-CD18, or anti-CD11b antibodies); growth factors such as VEGF-A, VEGF-C; tissue factor (TF); alpha interferon (alphaIFN); TNFalpha, an interleukin, such as IL-1 beta, IL-3, IL-4, IL-5, IL-6, IL-8, IL-9, IL-13, IL 17 AF, IL-1S, IL-13R alphal, IL13R alpha2, IL-4R, IL-5R, IL-9R, IgE; blood group antigens; flk2/flt3 receptor; obesity (OB) receptor; mpl receptor; CTLA-4; RANKL, RANK, RSV F protein, protein C etc.

In certain embodiments, the cells and methods provided herein can be used to produce an antibody (or a multispecific antibody, such as a bispecific antibody) that specifically binds to complement protein C5 (e.g., an anti-C5 agonist antibody that specifically binds to human C5). In certain embodiments, the anti-C5 antibody comprises 1, 2, 3, 4, 5 or 6 CDRs selected from (a) a heavy chain variable region CDR1 comprising the amino acid sequence of SSYYMA (SEQ ID NO:1); (b) a heavy chain variable region CDR2 comprising the amino acid sequence of AIFTGSGAEYKAEWAKG (SEQ ID NO:26); (c) a heavy chain variable region CDR3 comprising the amino acid sequence of DAGYDYPTHAMHY (SEQ ID NO: 27); (d) a light chain variable region CDR1 comprising the amino acid sequence of RASQGISSSLA (SEQ ID NO: 28); (e) a light chain variable region CDR2 comprising the amino acid sequence of GASETES (SEQ ID NO: 29); and (f) a light chain variable region CDR3 comprising the amino acid sequence of QNTKVGSSYGNT (SEQ ID NO: 30). For example, in certain embodiments, the anti-C5 antibody comprises a heavy chain variable domain (VH) sequence comprising one, two or three CDRs selected from: (a) a heavy chain variable region CDR1 comprising the amino acid sequence of (SSYYMA (SEQ ID NO: 1); (b) a heavy chain variable region CDR2 comprising the amino acid sequence of AIFTGSGAEYKAEWAKG (SEQ ID NO: 26); (c) a heavy chain variable region CDR3 comprising the amino acid sequence of DAGYDYPTHAMHY (SEQ ID NO: 27); and/or a light chain variable domain (VL) sequence comprising one, two or three CDRs selected from (d) a light chain variable region CDR1 comprising the amino acid sequence of RASQGISSSLA (SEQ ID NO: 28); (e) a light chain variable region CDR2 comprising the amino acid sequence of GASETES (SEQ ID NO: 29); and (f) a light chain variable region CDR3 comprising the amino acid sequence of QNTKVGSSYGNT (SEQ ID NO: 30). The sequences of CDR1, CDR2 and CDR3 of the heavy chain variable region and CDR1, CDR2 and CDR3 of the light chain variable region above are disclosed in US 2016/0176954 as SEQ ID NO: 117, SEQ ID NO: 118, SEQ ID NO: 121, SEQ ID NO: 122, SEQ ID NO: 123, and SEQ ID NO: 125, respectively. (See Tables 7 and 8 in US 2016/0176954.)

In certain embodiments, the anti-C5 antibody comprises the VH and VL sequences

(SEQ ID NO: 31)
QVQLVESGGG LVQPGRSLRL SCAASGFTVH SSYYMAWVRQ
APGKGLEWVG AIFTGSGAEY KAEWAKGRVT ISKDTSKNQV
VLTMTNMDPV DTATYYCASD AGYDYPTHAM HYWGQGTLVT
VSS
and
(SEQ ID NO: 9)
DIQMTQSPSS LSASVGDRVT ITCRASQDIS NYLNWYQQKP
GKAPKLLIYY TSRLRSGVPS RFSGSGSGTD FTLTISSLQP
EDFATYYCQQ GHTLPPTFGQ GTKVEIK,

respectively, including post-translational modifications of those sequences. The VH and VL sequences above are disclosed in US 2016/0176954 as SEQ ID NO: 106 and SEQ ID NO: 111, respectively (See Tables 7 and 8 in US 2016/0176954.) In certain embodiments, the anti-CS antibody is 305L015 (see US 2016/0176954).

In certain embodiments, an antibody produced by methods disclosed herein is capable of binding to OX40 (e.g., an anti-OX40 agonist antibody that specifically binds to human OX40). In certain embodiments, the anti-OX40 antibody comprises 1, 2, 3, 4, 5 or 6 CDRs selected from (a) a heavy chain variable region CDR1 comprising the amino acid sequence of DSYMS (SEQ ID NO: 2); (b) a heavy chain variable region CDR2 comprising the amino acid sequence of DMYPDNGDSSYNQKFRE (SEQ ID NO: 3); (c) a heavy chain variable region CDR3 comprising the amino acid sequence of APRWYFSV (SEQ ID NO: 4); (d) a light chain variable region CDR1 comprising the amino acid sequence of RASQDISNYLN (SEQ ID NO: 5); (e) a light chain variable region CDR2 comprising the amino acid sequence of YTSRLRS (SEQ ID NO: 6); and (f) a light chain variable region CDR3 comprising the amino acid sequence of QQGHTLPPT (SEQ ID NO: 7). For example, in certain embodiments, the anti-OX40 antibody comprises a heavy chain variable domain (VH) sequence comprising one, two or three CDRs selected from: (a) a heavy chain variable region CDR1 comprising the amino acid sequence of DSYMS (SEQ ID NO: 2); (b) a heavy chain variable region CDR2 comprising the amino acid sequence of DMYPDNGDSSYNQKFRE (SEQ ID NO: 3); and (c) a heavy chain variable region CDR3 comprising the amino acid sequence of APRWYFSV (SEQ ID NO: 4) and/or a light chain variable domain (VL) sequence comprising one, two or three CDRs selected from (a) a light chain variable region CDR1 comprising the amino acid sequence of RASQDISNYLN (SEQ ID NO: 5); (b) a light chain variable region CDR2 comprising the amino acid sequence of YTSRLRS (SEQ ID NO: 6); and (c) a light chain variable region CDR3 comprising the amino acid sequence of QQGHTLPPT (SEQ ID NO: 7). In certain embodiments, the anti-OX40 antibody comprises the VH and VL sequences

(SEQ ID NO: 8)
EVQLVQSGAE VKKPGASVKV SCKASGYTFT DSYMSWVRQA
PGQGLEWIGD MYPDNGDSSY NQKFRERVTI TRDTSTSTAY
LELSSLRSED TAVYYCVLAP RWYFSVWGQG TLVTVSS
and
(SEQ ID NO: 9)
DIQMTQSPSS LSASVGDRVT ITCRASQDIS NYLNWYQQKP
GKAPKLLIYY TSRLRSGVPS RFSGSGSGTD FTLTISSLQP
EDFATYYCQQ GHTLPPTFGQ GTKVEIK,

respectively, including post-translational modifications of those sequences.

In certain embodiments, the anti-OX40 antibody comprises 1, 2, 3, 4, 5 or 6 CDRs selected from (a) a heavy chain variable region CDR1 comprising the amino acid sequence of NYLIE (SEQ ID NO: 10); (b) a heavy chain variable region CDR2 comprising the amino acid sequence of VINPGSGDTYYSEKFKG (SEQ ID NO: 11); (c) a heavy chain variable region CDR3 comprising the amino acid sequence of DRLDY (SEQ ID NO: 12); (d) a light chain variable region CDR1 comprising the amino acid sequence of HASQDISSYIV (SEQ ID NO: 13); (e) a light chain variable region CDR2 comprising the amino acid sequence of HGTNLED (SEQ ID NO: 14); and (f) a light chain variable region CDR3 comprising the amino acid sequence of VHYAQFPYT (SEQ ID NO: 15). For example, in certain embodiments, the anti-OX40 antibody comprises a heavy chain variable domain (VH) sequence comprising one, two or three CDRs selected from: (a) a heavy chain variable region CDR1 comprising the amino acid sequence of NYLIE (SEQ ID NO: 10); (b) a heavy chain variable region CDR2 comprising the amino acid sequence of VINPGSGDTYYSEKFKG (SEQ ID NO: 11); and (c) a heavy chain variable region CDR3 comprising the amino acid sequence of DRLDY (SEQ ID NO: 12) and/or a light chain variable domain (VL) sequence comprising one, two or three CDRs selected from (a) a light chain variable region CDR1 comprising the amino acid sequence of HASQDISSYIV (SEQ ID NO: 13); (b) a light chain variable region CDR2 comprising the amino acid sequence of HGTNLED (SEQ ID NO: 14); and (c) a light chain variable region CDR3 comprising the amino acid sequence of VHYAQFPYT (SEQ ID NO: 15). In certain embodiments, the anti-OX40 antibody comprises the VH and VL EVQLVQSGAE VKKPGASVKV SCKASGYAFT NYLIEWVRQA PGQGLEWIGV INPGSGDTYY SEKFKGRVTI TRDTSTSTAY LELSSLRSED TAVYYCARDR LDYWGQGTLV TVSS (SEQ ID NO: 16) and DIQMTQSPSS LSASVGDRVT ITCHASQDIS SYIVWYQQKP GKAPKLLIYH GTNLEDGVPS RFSGSGSGTD FTLTISSLQP EDFATYYCVH YAQFPYTFGQ GTKVEIK (SEQ ID NO: 17), respectively, including post-translational modifications of those sequences.

Further details regarding anti-OX40 antibodies are provided in WO 2015/153513, which is incorporated herein by reference in its entirety.

In certain embodiments, an antibody produced by the cells and methods disclosed herein is capable of binding to influenza virus B hemagglutinin, i.e., “fluB” (e.g., an antibody that binds hemagglutinin from the Yamagata lineage of influenza B viruses, binds hemagglutinin from the Victoria lineage of influenza B viruses, binds hemagglutinin from ancestral lineages of influenza B virus, or binds hemagglutinin from the Yamagata lineage, the Victoria lineage, and ancestral lineages of influenza B virus, in vitro and/or in vivo). Further details regarding anti-FluB antibodies are described in WO 2015/148806, which is incorporated herein by reference in its entirety.

In certain embodiments, an antibody produced by the cells and methods disclosed herein is capable of binding to low density lipoprotein receptor-related protein (LRP)-1 or LRP-8 or transferrin receptor, and at least one target selected from the group consisting of beta-secretase (BACE1 or BACE2), alpha-secretase, gamma-secretase, tau-secretase, amyloid precursor protein (APP), death receptor 6 (DR6), amyloid beta peptide, alpha-synuclein, Parkin, Huntingtin, p75 NTR, CD40 and caspase-6.

In certain embodiments, an antibody produced by the cells and methods disclosed herein is a human IgG2 antibody against CD40. In certain embodiments, the anti-CD40 antibody is RG7876.

In certain embodiments, the cells and methods of the present disclosure can be used to product a polypeptide. For example, but not by way of limitation, the polypeptide is a targeted immunocytokine. In certain embodiments, the targeted immunocytokine is a CEA-IL2v immunocytokine. In certain embodiments, the CEA-IL2v immunocytokine is RG7813. In certain embodiments, the targeted immunocytokine is a FAP-IL2v immunocytokine. In certain embodiments, the FAP-IL2v immunocytokine is RG7461.

In certain embodiments, the multispecific antibody (such as a bispecific antibody) produced by the cells or methods provided herein is capable of binding to CEA and at least one additional target molecule. In certain embodiments, the multispecific antibody (such as a bispecific antibody) produced according to methods provided herein is capable of binding to a tumor targeted cytokine and at least one additional target molecule. In certain embodiments, the multispecific antibody (such as a bispecific antibody) produced according to methods provided herein is fused to IL2v (i.e., an interleukin 2 variant) and binds an IL1-based immunocytokine and at least one additional target molecule. In certain embodiments, the multispecific antibody (such as a bispecific antibody) produced according to methods provided herein is a T-cell bispecific antibody (i.e., a bispecific T-cell engager or BiTE).

In certain embodiments, the multispecific antibody (such as a bispecific antibody) produced according to methods provided herein is capable of binding to at least two target molecules selected from: IL-1 alpha and IL-1 beta, IL-12 and IL-1S; IL-13 and IL-9; IL-13 and IL-4; IL-13 and IL-5; IL-5 and IL-4; IL-13 and IL-1beta; IL-13 and IL-25; IL-13 and TARC; IL-13 and MDC; IL-13 and MEF; IL-13 and TGF-˜; IL-13 and LHR agonist; IL-12 and TWEAK, IL-13 and CL25; IL-13 and SPRR2a; IL-13 and SPRR2b; IL-13 and ADAMS, IL-13 and PED2, IL17A and IL17F, CEA and CD3, CD3 and CD19, CD138 and CD20; CD138 and CD40; CD19 and CD20; CD20 and CD3; CD3S and CD13S; CD3S and CD20; CD3S and CD40; CD40 and CD20; CD-S and IL-6; CD20 and BR3, TNF alpha and TGF-beta, TNF alpha and IL-1 beta; TNF alpha and IL-2, TNF alpha and IL-3, TNF alpha and IL-4, TNF alpha and IL-5, TNF alpha and IL6, TNF alpha and IL8, TNF alpha and IL-9, TNF alpha and IL-10, TNF alpha and IL-11, TNF alpha and IL-12, TNF alpha and IL-13, TNF alpha and IL-14, TNF alpha and IL-15, TNF alpha and IL-16, TNF alpha and IL-17, TNF alpha and IL-18, TNF alpha and IL-19, TNF alpha and IL-20, TNF alpha and IL-23, TNF alpha and IFN alpha, TNF alpha and CD4, TNF alpha and VEGF, TNF alpha and MIF, TNF alpha and ICAM-1, TNF alpha and PGE4, TNF alpha and PEG2, TNF alpha and RANK ligand, TNF alpha and Te38, TNF alpha and BAFF,TNF alpha and CD22, TNF alpha and CTLA-4, TNF alpha and GP130, TNF a and IL-12p40, VEGF and Angiopoietin, VEGF and HER2, VEGF-A and HER2, VEGF-A and PDGF, HER1 and HER2, VEGFA and ANG2,VEGF-A and VEGF-C, VEGF-C and VEGF-D, HER2 and DR5,VEGF and IL-8, VEGF and MET, VEGFR and MET receptor, EGFR and MET, VEGFR and EGFR, HER2 and CD64, HER2 and CD3, HER2 and CD16, HER2 and HER3; EGFR (HER1) and HER2, EGFR and HER3, EGFR and HER4, IL-14 and IL-13, IL-13 and CD40L, IL4 and CD40L, TNFR1 and IL-1 R, TNFR1 and IL-6R and TNFR1 and IL-18R, EpCAM and CD3, MAPG and CD28, EGFR and CD64, CSPGs and RGM A; CTLA-4 and BTN02; IGF1 and IGF2; IGF1/2 and Erb2B; MAG and RGM A; NgR and RGM A; NogoA and RGM A; OMGp and RGM A; POL-1 and CTLA-4; and RGM A and RGM B.

In certain embodiments, a multispecific antibody (such as a bispecific antibody) produced by the cells and methods disclosed herein is an anti-VEGF/anti-angiopoietin bispecific antibody. In certain embodiments, the anti-VEGF/anti-angiopoietin bispecific antibody bispecific antibody is a Crossmab. In certain embodiments, the anti-VEGF/anti-angiopoietin bispecific antibody is RG7716.

In certain embodiments, the multispecific antibody (such as a bispecific antibody) produced by methods disclosed herein is an anti-Ang2/anti-VEGF bispecific antibody. In certain embodiments, the anti-Ang2/anti-VEGF bispecific antibody is RG7221. In certain embodiments, the anti-Ang2/anti-VEGF bispecific antibody is CAS Number 1448221-05-3.

Soluble antigens or fragments thereof, optionally conjugated to other molecules, can be used as immunogens for generating antibodies. For transmembrane molecules, such as receptors, fragments of these (e.g., the extracellular domain of a receptor) can be used as the immunogen. Alternatively, cells expressing the transmembrane molecule can be used as the immunogen. Such cells can be derived from a natural source (e.g., cancer cell lines) or can be cells which have been transformed by recombinant techniques to express the transmembrane molecule. Other antigens and forms thereof useful for preparing antibodies will be apparent to those in the art.

In certain embodiments, the polypeptide (e.g., antibodies) produced by the cells and methods disclosed herein is capable of binding to can be further conjugated to a chemical molecule such as a dye or cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). An immunoconjugate comprising an antibody or bispecific antibody produced using the methods described herein can contain the cytotoxic agent conjugated to a constant region of only one of the heavy chains or only one of the light chains.

### 5.5.6 Antibody Variants

In certain aspects, amino acid sequence variants of the antibodies provided herein are contemplated, e.g., the antibodies provided in Section 5.5.5. For example, it can be desirable to alter the binding affinity and/or other biological properties of the antibody. Amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### 5.5.6.1 Substitution, Insertion, and Deletion Variants

In certain aspects, antibody variants having one or more amino acid substitutions are provided. Sites of interest for substitutional mutagenesis include the CDRs and FRs. Conservative substitutions are shown in Table 1 under the heading of “preferred substitutions”. More substantial changes are provided in Table 1 under the heading of “exemplary substitutions”, and as further described below in reference to amino acid side chain classes. Amino acid substitutions can be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids can be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which can be conveniently generated, e.g., using phage display-based affinity maturation techniques such as those described herein. Briefly, one or more. CDR residues are mutated and the variant antibodies displayed on phage and screened for a particular biological activity (e.g., binding affinity).

Alterations (e.g., substitutions) can be made in CDRs, e.g., to improve antibody affinity. Such alterations can be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding can be specifically identified, e.g., using alanine scanning mutagenesis or modeling. CDR-H3 and CDR-L3 in particular are often targeted.

In certain aspects, substitutions, insertions, or deletions can occur within one or more CDRs so long as such alterations do not substantially reduce the ability of the antibody to bind antigen. For example, conservative alterations (e.g., conservative substitutions as provided herein) that do not substantially reduce binding affinity can be made in the CDRs. Such alterations can, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that can be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions can be introduced at the amino acid locations demonstrating functional sensitivity to the initial substitutions. Alternatively, or additionally, a crystal structure of an antigen-antibody complex can be used to identify contact points between the antibody and antigen. Such contact residues and neighboring residues can be targeted or eliminated as candidates for substitution. Variants can be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### 5.5.6.2 Glycosylation Variants

In certain aspects, an antibody provided herein is altered to increase or decrease the extent to which the antibody is glycosylated. Addition or deletion of glycosylation sites to an antibody can be conveniently accomplished by altering the amino acid sequence such that one or more glycosylation sites is created or removed.

Where the antibody comprises an Fc region, the oligosaccharide attached thereto can be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide can include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the disclosure can be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides can be at least about 20%, at least about 40%, at least about 60%, at least about 80%, or even about 100% (i.e., no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 can also be located about ±3 amino acids upstream or downstream of position 297, i.e., between positions 294 and 300, due to minor sequence variations in antibodies. Such antibodies having an increased proportion of non-fucosylated oligosaccharides in the Fc region can have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants can have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants can have improved CDC function. Such antibody variants are described, e.g., in WO 1997/30087; WO 1998/58964; and WO 1999/22764.

### 5.5.6.3 Fe Region Variants

In certain aspects, one or more amino acid modifications can be introduced into the Fc region of an antibody provided herein, thereby generating an Fc region variant. The Fc region variant can comprise a human Fc region sequence (e.g., a human IgG₁, IgG₂, IgG₃ or IgG₄ Fc region) comprising an amino acid modification (e.g., a substitution) at one or more amino acid positions.

In certain aspects, the present disclosure contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods can be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest can be assessed in vivo, e.g., in a animal model such as that disclosed in Clynes et al. Proc. Nat'l Acad Sci. USA 95:652-656 (1998). C1q binding assays can also be carried out to confirm that the antibody is unable to bind C1q and hence lacks CDC activity. See, e.g., C1q and C3c binding ELISA in WO 2006/029879 and WO 2005/100402. To assess complement activation, a CDC assay can be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S.B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG₁ Fc region. In one aspect, the substitutions are L234A, L235A and P329G (LALA-PG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In some aspects, alterations are made in the Fc region that result in altered (i.e., either improved or diminished) C1q binding and/or Complement Dependent Cytotoxicity

(CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues 1253, H310, H433, N434, and H435 (EU index numbering) are involved in the interaction (Medesan, C., et al., Eur. J. Immunol. 26 (1996) 2533; Firan, M., et al., Int. Immunol. 13 (2001) 993; Kim, J.K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, 5254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 A1).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### 5.5.6.4 Cysteine Engineered Antibody Variants

In certain aspects, it can be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and can be used to conjugate the antibody to other moieties, such as drug moieties or linker-drug moieties, to create an immunoconjugate, as described further herein. Cysteine engineered antibodies can be generated as described, e.g., in U.S. Pat. Nos. 7,521,541, 8,30,930, 7,855,275, 9,000,130, or WO 2016040856.

### 5.5.6.5 Antibody Derivatives

In certain aspects, an antibody provided herein can be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde can have advantages in manufacturing due to its stability in water. The polymer can be of any molecular weight, and can be branched or unbranched. The number of polymers attached to the antibody can vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### 5.5.7 Immunoconjugates

The present disclosure also provides immunoconjugates comprising an antibody disclosed herein conjugated (chemically bonded) to one or more therapeutic agents such as cytotoxic agents, chemotherapeutic agents, drugs, growth inhibitory agents, toxins (e.g., protein toxins, enzymatically active toxins of bacterial, fungal, plant, or animal origin, or fragments thereof), or radioactive isotopes.

In one aspect, an immunoconjugate is an antibody-drug conjugate (ADC) in which an antibody is conjugated to one or more of the therapeutic agents mentioned above. The antibody is typically connected to one or more of the therapeutic agents using linkers. An overview of ADC technology including examples of therapeutic agents and drugs and linkers is set forth in Pharmacol Review 68:3-19 (2016).

In another aspect, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another aspect, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re₁₈₈, Sm¹⁵³, Bi²¹², P³², Pb²¹² and radioactive isotopes of Lu. When the radioconjugate is used for detection, it can comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent can be made using a variety of bifunctional protein coupling agents such as N-succinimidyl-3-(2-pyridyldithio) propionate (SPDP), succinimidyl-4-(N-maleimidomethyl) cyclohexane-l-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO 94/11026. The linker can be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) can be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### EXEMPLARY EMBODIMENTS

A. The presently described subject matter provides a method of producing a cell comprising edits at two or more target loci:
combining two or more guide RNAs (gRNAs) capable of directing CRISPR/Cas9-mediated indel formation at respective target loci with Cas9 protein to form a ribonucleoprotein complex (RNP);
serially transfecting a population of cells with the RNP until at least about 10% indel formation is achieved at each target locus; and
isolating a cell comprising edits at two or more target loci by single cell cloning of the cell from the population of serially transfected cells.

A1. The foregoing method of A, wherein the gRNA is an sgRNA.

A2. The foregoing method of A, wherein the gRNA comprises a crRNA and a tracrRNA.

A3. The foregoing method of A2, wherein the crRNA is an XT-gRNA.

A4. The foregoing method of any one of A-A3, wherein the population of cells is serially transfected with the RNP until at least about 20% indel formation is achieved at each target locus.

A5. The foregoing method of any one of A-A3, wherein the population of cells is serially transfected with the RNP until at least about 30% indel formation is achieved at each target locus.

A6. The foregoing method of any one of A-A3, wherein the population of cells is serially transfected with the RNP until at least about 40% indel formation is achieved at each target locus.

A7. The foregoing method of any one of A-A3, wherein the population of cells is serially transfected with the RNP until at least about 50% indel formation is achieved at each target locus.

A8. The foregoing method of any one of A-A3, wherein the population of cells is serially transfected with the RNP until at least about 60% indel formation is achieved at each target locus.

A9. The foregoing method of A, wherein the ratio of moles of RNP to number of transfected cells is between about 0.1 pmol per 10⁶ cells to about 5 pmol per 10⁶ cells

A10. The foregoing method of A, wherein the ratio of moles of RNP to number of transfected cells is about 0.15 pmol per 10⁶ cells.

A11. The foregoing method of A, wherein the ratio of moles of RNP to number of transfected cells is about 0.17 pmol per 10⁶ cells.

A12. The foregoing method of A, wherein the ratio of moles of RNP to number of transfected cells is about 0.2 pmol per 10⁶ cells.

A13. The foregoing method of A, wherein the ratio of moles of RNP to number of transfected cells is about 1 pmol per 10⁶ cells.

A14. The foregoing method of A, wherein the ratio of moles of RNP to number of transfected cells is about 2 pmol per 10⁶ cells.

A15. The foregoing method of A, wherein the ratio of moles of RNP to number of transfected cells is about 3 pmol per 10⁶ cells.

A16. The foregoing method of A, wherein three or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

A17. The foregoing method of A, wherein four or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

A18. The foregoing method of A, wherein five or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

A19. The foregoing method of A, wherein six or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

A20. The foregoing method of A, wherein seven or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

A21. The foregoing method of A, wherein eight or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

A22. The foregoing method of A, wherein nine or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

A23. The foregoing method of A, wherein ten or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

A24. The foregoing method of any one of A16-A23, wherein the RNPs are serially transfecting into a population of cells until at least about 20% indel formation is achieved at each target locus.

A25. The foregoing method of any one of A16-A23, wherein the RNPs are serially transfecting into a population of cells until at least about 20% indel formation is achieved at each target locus.

A26. The foregoing method of any one of A16-A23, wherein the RNPs are serially transfecting into a population of cells until at least about 30% indel formation is achieved at each target locus.

A27. The foregoing method of any one of A16-A23, wherein the RNPs are serially transfecting into a population of cells until at least about 40% indel formation is achieved at each target locus.

A28. The foregoing method of any one of A16-A23, wherein the RNPs are serially transfecting into a population of cells until at least about 50% indel formation is achieved at each target locus.

A29. The foregoing method of any one of A16-A23, wherein the RNPs are serially transfecting into a population of cells until at least about 60% indel formation is achieved at each target locus.

A30. The foregoing method of any one of A-A29, wherein the cell is a T cell, an NK cell, a B cell, a dendritic cell, a CHO cell, a COS-7 cell; an HEK 293 cell, a BHK cells, a TM4 cell, a CV1 cell; a VERO-76 cell; a HELA cells; or an MDCK cell.

A31. The foregoing method of A, wherein the two or more gRNAs capable of directing CRISPR/Cas9-mediated indel formation at respective target loci are identified via a efficiency screen comprising:
transfecting a population of cells with a population of RNPs, where each RNP comprises a gRNA capable of directing CRISPR/Cas9-mediated indel formation at a target locus; and
sequencing the target loci to identify gRNAs based on their efficiency in directing CRISPR/Cas9-mediated indel formation.

A32. The foregoing method of A31, wherein the sequencing is performed using Sanger sequencing.

B. The presently described subject matter provides a cell composition, wherein the cell comprises edits at two or more target loci, wherein the edits are the result of:
combining two or more gRNAs capable of directing CRISPR/Cas9-mediated indel formation at respective target loci with Cas9 protein to form an RNP;
serially transfecting a population of cells with the RNP until at least about 10% indel formation is achieved at each target locus; and
isolating the cell comprising edits at two or more target loci by single cell cloning of the cell from the population of serially transfected cells

C. The presently described subject matter provides a cell composition, host cell composition, wherein the host cell comprises:
a nucleic acid encoding a non-endogenous polypeptide of interest; and
edits at two more target loci, wherein the edits are the result of:
   combining two or more gRNAs capable of directing CRISPR/Cas9-mediated indel formation at respective target loci with Cas9 protein to form an RNP;
   serially transfecting a population of cells with the RNP until at least about 10% indel formation is achieved at each target locus; and
   isolating the host cell comprising edits at two or more target loci by single cell cloning of the host cell from the population of serially transfected cells.

D. The cell composition of B or the host cell composition of C, wherein the gRNA is an sgRNA.

D1. The cell composition of B or the host cell composition of C, wherein the gRNA comprises a crRNA and a tracrRNA.

D2. The cell composition of D1 or the host cell composition of D1, wherein the crRNA is an XT-gRNA.

D3. The cell composition of B or the host cell composition of C, wherein the population of cells is serially transfected with the RNP until at least about 20% indel formation is achieved at each target locus.

D4. The cell composition of B or the host cell composition of C, wherein the population of cells is serially transfected with the RNP until at least about 30% indel formation is achieved at each target locus.

D5. The cell composition of B or the host cell composition of C, wherein the population of cells is serially transfected with the RNP until at least about 40% indel formation is achieved at each target locus.

D6. The cell composition of B or the host cell composition of C, wherein the population of cells is serially transfected with the RNP until at least about 50% indel formation is achieved at each target locus.

D7. The cell composition of B or the host cell composition of C, wherein the population of cells is serially transfected with the RNP until at least about 60% indel formation is achieved at each target locus.

D8. The cell composition of B or the host cell composition of C, wherein the ratio of moles of RNP to number of transfected cells is between about 0.1 pmol per 10⁶ cells to about 5 pmol per 10⁶ cells

D9. The cell composition of B or the host cell composition of C, wherein the ratio of moles of RNP to number of transfected cells is about 0.15 pmol per 10⁶ cells.

D10. The cell composition of B or the host cell composition of C, wherein the ratio of moles of RNP to number of transfected cells is about 0.17 pmol per 10⁶ cells.

D11. The cell composition of B or the host cell composition of C, wherein the ratio of moles of RNP to number of transfected cells is about 0.2 pmol per 10⁶ cells.

D12. The cell composition of B or the host cell composition of C, wherein the ratio of moles of RNP to number of transfected cells is about 1 pmol per 10⁶ cells.

D13. The cell composition of B or the host cell composition of C, wherein the ratio of moles of RNP to number of transfected cells is about 2 pmol per 10⁶ cells.

D14. The cell composition of B or the host cell composition of C, wherein the ratio of moles of RNP to number of transfected cells is about 3 pmol per 10⁶ cells.

D15. The cell composition of B or the host cell composition of C, wherein three or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

D16. The cell composition of B or the host cell composition of C, wherein four or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

D17. The cell composition of B or the host cell composition of C, wherein five or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

D18. The cell composition of B or the host cell composition of C, wherein six or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

D19. The cell composition of B or the host cell composition of C, wherein seven or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

D20. The cell composition of B or the host cell composition of C, wherein eight or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

D21. The cell composition of B or the host cell composition of C, wherein nine or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

D22. The cell composition of B or the host cell composition of C, wherein ten or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

D23. The cell composition or the host cell composition of any of D15-D22, wherein the RNPs are serially transfecting into a population of cells until at least about 20% indel formation is achieved at each target locus.

D24. The cell composition or the host cell composition of any of D15-D22, wherein the RNPs are serially transfecting into a population of cells until at least about 20% indel formation is achieved at each target locus.

D24. The cell composition or the host cell composition of any of D15-D22, wherein the RNPs are serially transfecting into a population of cells until at least about 30% indel formation is achieved at each target locus.

D25. The cell composition or the host cell composition of any of D15-D22, wherein the RNPs are serially transfecting into a population of cells until at least about 40% indel formation is achieved at each target locus.

D26. The cell composition or the host cell composition of any of D15-D22, wherein the RNPs are serially transfecting into a population of cells until at least about 50% indel formation is achieved at each target locus.

D27. The cell composition or the host cell composition of any of D15-D22, wherein the RNPs are serially transfecting into a population of cells until at least about 60% indel formation is achieved at each target locus.

D28. The cell composition or the host cell composition of any of D-D22, wherein the cell is a T cell, an NK cell, a B cell, a dendritic cell, a CHO cell, a COS-7 cell; an HEK 293 cell, a BHK cells, a TM4 cell, a CV1 cell; a VERO-76 cell; a HELA cells; or an MDCK cell.

D29. The cell composition of B or the host cell composition of C, wherein the two or more gRNAs capable of directing CRISPR/Cas9-mediated indel formation at respective target loci are identified via an efficiency screen comprising:
transfecting a population of cells with a population of RNPs, where each RNP comprises a gRNA capable of directing CRISPR/Cas9-mediated indel formation at a target locus; and
sequencing the target loci to identify gRNAs based on their efficiency in directing CRISPR/Cas9-mediated indel formation.

D23. The cell composition or the host cell composition of D29, wherein the sequencing is performed using Sanger sequencing.

E. The presently described subj ect matter provides a method producing a polypeptide of interest comprising:
culturing a host cell composition comprising:
   a nucleic acid encoding a non-endogenous polypeptide of interest; and
   edits at two or more target loci, wherein the edits are the result of:
      combining two or more gRNAs capable of directing CRISPR/Cas9-mediated indel formation at respective target loci with Cas9 protein to form an RNP;
      serially transfecting a population of cells with the RNP until about 10% indel formation is achieved at each target locus; and
      isolating the host cell comprising edits at two or more target loci by single cell cloning of the host cell from the population of serially transfected cells; and
isolating the polypeptide of interest expressed by the cultured host cell.

E1. The method of E, wherein the gRNA is an sgRNA.

E2. The method of E, wherein the gRNA comprises a crRNA and a tracrRNA.

E3. The method of E2, wherein the crRNA is an XT-gRNA.

E4. The method of any of E-E3, wherein the population of cells is serially transfected with the RNP until at least about 20% indel formation is achieved at each target locus.

E5. The method of any of E-E3, wherein the population of cells is serially transfected with the RNP until at least about 30% indel formation is achieved at each target locus.

E6. The method of any of E-E3, wherein the population of cells is serially transfected with the RNP until at least about 40% indel formation is achieved at each target locus.

E7. The method of any of E-E3, wherein the population of cells is serially transfected with the RNP until at least about 50% indel formation is achieved at each target locus.

E8. The method of any of E-E3, wherein the population of cells is serially transfected with the RNP until at least about 60% indel formation is achieved at each target locus.

E9. The method of E, wherein the ratio of moles of RNP to number of transfected cells is between about 0.1 pmol per 10⁶ cells to about 5 pmol per 10⁶ cells

E10. The method of E, wherein the ratio of moles of RNP to number of transfected cells is about 0.15 pmol per 10⁶ cells.

E11. The method of E, wherein the ratio of moles of RNP to number of transfected cells is about 0.17 pmol per 10⁶ cells.

E12. The method of E, wherein the ratio of moles of RNP to number of transfected cells is about 0.2 pmol per 10⁶ cells.

E13. The method of E, wherein the ratio of moles of RNP to number of transfected cells is about 1 pmol per 10⁶ cells.

E14. The method of E, wherein the ratio of moles of RNP to number of transfected cells is about 2 pmol per 10⁶ cells.

E15. The method of E, wherein the ratio of moles of RNP to number of transfected cells is about 3 pmol per 10⁶ cells.

E16. The method of E, wherein three or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

E17. The method of E, wherein four or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

E18. The method of E, wherein five or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

E19. The method of E, wherein six or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

E20. The method of E, wherein seven or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

E21. The method of E, wherein eight or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

E22. The method of E, wherein nine or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

E23. The method of E, wherein ten or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

E24. The method of any one of E16-E23, wherein the RNPs are serially transfecting into a population of cells until at least about 20% indel formation is achieved at each target locus.

E25. The method of any one of E16-E23, wherein the RNPs are serially transfecting into a population of cells until at least about 20% indel formation is achieved at each target locus.

E26. The method of any one of E16-E23, wherein the RNPs are serially transfecting into a population of cells until at least about 30% indel formation is achieved at each target locus.

E27. The method of any one of E16-E23, wherein the RNPs are serially transfecting into a population of cells until at least about 40% indel formation is achieved at each target locus.

E28. The method of any one of E16-E23, wherein the RNPs are serially transfecting into a population of cells until at least about 50% indel formation is achieved at each target locus.

E29. The method of any one of E16-E23, wherein the RNPs are serially transfecting into a population of cells until at least about 60% indel formation is achieved at each target locus.

E30. The method of any one of E-E29, wherein the cell is a T cell, an NK cell, a B cell, a dendritic cell, a CHO cell, a COS-7 cell; an HEK 293 cell, a BHK cells, a TM4 cell, a CV1 cell; a VERO-76 cell; a HELA cells; or an MDCK cell.

E31. The method of E, wherein the two or more gRNAs capable of directing CRISPR/Cas9-mediated indel formation at respective target loci are identified via a efficiency screen comprising:
a. transfecting a population of cells with a population of RNPs, where each RNP comprises a gRNA capable of directing CRISPR/Cas9-mediated indel formation at a target locus; and
b. sequencing the target loci to identify gRNAs based on their efficiency in directing CRISPR/Cas9-mediated indel formation.

E32. The method of E31, wherein the sequencing is performed using Sanger sequencing.

E33. The method of any one of E-E32, wherein the method comprises purifying the product of interest, harvesting the product of interest, and/or formulating the product of interest.

E34. The method of any one of E-E32, wherein wherein the cell is a mammalian cell.

E35. The method of E34, wherein the mammalian cell is a CHO cell.

E36. The method of any one of E-E32, wherein polypeptide of interest comprises an antibody or an antigen-binding fragment thereof.

E37. The method of E36, wherein the antibody is a multispecific antibody or an antigen-binding fragment thereof.

E38. The method of E36, wherein the antibody consists of a single heavy chain sequence and a single light chain sequence or antigen-binding fragments thereof.

E39. The method of E36, wherein the antibody is a chimeric antibody, a human antibody or a humanized antibody.

E40. The method of E36, wherein the antibody is a monoclonal antibody.

F. The host cell composition of C, wherein polypeptide of interest comprises an antibody or an antigen-binding fragment thereof.

F1. The host cell composition of F, wherein the antibody is a multispecific antibody or an antigen-binding fragment thereof.

F1. The host cell composition of F, wherein the antibody consists of a single heavy chain sequence and a single light chain sequence or antigen-binding fragments thereof.

F1. The host cell composition of F, wherein the antibody is a chimeric antibody, a human antibody or a humanized antibody.

F1. The host cell composition of F, wherein the antibody is a monoclonal antibody.

### EXAMPLES

The following examples are merely illustrative of the presently disclosed subject matter and should not be considered as limitations in any way.

### Materials and Methods

### Cell Culture

Parental and KO host CHO cell lines were maintained as previously described. (Carver et al., Biotechnology Progress. 2020:e2967). Briefly, CHO cells were cultured in a proprietary DMEM/F12-based medium in 125 mL shake flask vessels maintained at 150 rpm agitation, 37° C., and 5% CO₂. Cells were passaged at a seeding density of 4×10⁵ cells/mL every 3-4 days.

Fed-batch production cultures were performed for the 6× KO and 10× KO clones in shake flasks using proprietary chemically defined medium with bolus nutrient feeds on days 3, 6, 8, and 10 as previously described (Ko et al., Biotechnology Progress. 2018;34(3):624-634). Viable cell count (VCD) was measured throughout the experiment using a Vi-Cell XR instrument (Beckman Coulter). Integrated viable cell count (IVCC) for each production culture was calculated using VCD measurements; IVCC represents the integral of the area under the growth curve over the culture duration.

### Synthetic gRNA Target Design and Screening

The gene targets used in the 6× and 10× KO cell lines are listed in Table 2. gRNA sequences were designed using the CRISPR Guide RNA Design software (Benchling) and manufactured by Integrated DNA Technologies (IDT). gRNA sequences were selected based on the software's on and off-target scoring, and at least three gRNAs targeting an early exon were screened for each gene target.

The following reagents were used from IDT for the screening of gRNAs: Alt-R® CRISPR-Cas9 crRNA (crRNA), Alt-R® CRISPR-Cas9 crRNA XT (XT-gRNA), Alt-R® CRISPR-Cas9 tracrRNA (tracrRNA), and Alt-R® S.p. Cas9 Nuclease V3. RNPs were complexed together, 20 pmol crRNA or XT-gRNA annealed to 20 pmol of tracrRNA, combined with 20 pmol of Cas9 protein at a 1:1:1 ratio. RNPs were transfected into twelve million CHO cells using a Neon™ Transfection System and Neon™ Transfection System 100 μL Kit (Thermo Fisher Scientific). Transfection parameters were set to 1610 V, 10 ms pulse width, and 3 pulses.

### Genomic DNA PCR and gRNA Indel Analysis

At 48-72 hours post-transfection, DNA from RNP-transfected cells was extracted using the DNeasy Blood and Tissue Kit (Qiagen), and a 400-500 bp region of DNA centered on each gRNA cut site was PCR amplified. Amplicons were purified using the QlAquick PCR Purification Kit (Qiagen) and Sanger sequenced. The Sanger sequencing traces for each test sample and its corresponding control sample were uploaded to the Inference of CRISPR Edits (ICE) software tool and analyzed according to the developer's instructions (synthego.com/guide/how-to-use-crispr/ice-analysis-guide). ICE analysis reports indel percentage and “knockout score.” Indel percentage represents the editing efficiency of the edited trace against the control trace, regardless whether the indel results in a frameshift; the knockout score represents the proportion of cells that have either a frameshift indel or a fragment deletion (of 21+ bp), which likely results in a functional knockout. The gRNA with the highest knockout score for a particular target was selected to move forward into multiplexing experiments.

### TA Cloning and Western Blot Analysis

Transfected samples were analyzed by TA cloning to verify indel quantification by ICE analysis for target genes C-E. Briefly, the PCR product generated from the same PCR reaction for ICE analysis was ligated into the TA Cloning® Kit with pCR™ 2.1 vector (ThermoFisher Scientific). The ligation mixture was transformed into One Shot® TOP10 chemically competent E. coli (ThermoFisher Scientific). Plasmid DNA was isolated from single cell colonies and sequenced. Indel analysis for each gRNA was performed by manually examining the sequencing traces on a software (Sequencher).

Western blot was performed to confirm knockout efficiency for a target gene B. Five million cells were lysed 96 hours after RNP electroporation of two gRNAs. Protein concentration in the lysate was quantified, and equal total proteins were loaded, separated by electrophoresis, and blotted using standard techniques. Actin staining was used as the loading control.

### DNA Sequencing and ICE Analysis of Knockout Cell Pools and Single Cell Clone

Genomic DNA was extracted from transfected pools or single cell clones using the MagNA Pure 96 Instrument (Roche Life Science), followed by PCR to amplify the genomic region around each gRNA cut site as described previously. PCR products were then purified using the QIAquick 96 PCR purification kit (Qiagen) or the ZR-96 DNA Clean-Up Kit (Zymo Research) according to the manufacturer's instructions, followed by Sanger sequencing and ICE indel analysis. For 6× KO and 10× KO multiplex knockout experiments, a total of 496 clones and 704 single cell clones were screened respectively.

### Targeted Liquid Chromatography Followed by Tandem Mass Spectrometry (LC-MS/MS) Analysis for Confirmation of Gene Knockouts

On day 12 or 13 of production cultures for knockout cell lines, harvested cell culture fluid (HCCF) was obtained by centrifuging culture samples at 1000 RPM for 5 min and stored at −80° C. until sample preparation. Samples were equilibrated to room temperature for 30 min before use and diluted in purified water. Each diluted sample (100 ul) was added to a microcentrifuge tube and mixed with 400 ul of denaturation buffer (7.2M Guanidine hydrochloride, 0.3M sodium acetate, pH 5.0±0.1) and 10 ul of TCEP stock solution (0.5 M Bond-Breaker Tris(2-carboxyethyl)phosphine (TCEP), neutral pH). Water bath incubation of the samples was held at 37° C. for 15 min for reduction followed by the addition of 500 ul of the reduced sample to NAP-5 desalting columns. After elution and pH adjustment of the column, samples were digested by 0.5 mg/ml trypsin (20 ul) and incubated at 37° C. for 60 min. Reverse phase UPLC was used to analyze the samples. 1D LC-MS/MS targeted method was run on QTRAP, monitoring 3 peptides for target protein compared with an internal spike-in control (Bovine Carbonic Anhydrase; CA II). Positive protein identification requires at least 2 targeted peptides to be present.

### Example 1: Multiplex CRISPR Editing and Generation of 6× KO and 10× KO Cell Pools and Single Cell Clones

For the 6× KO (genes C, E-G, and J-K) and 10× KO (genes A-B and D-K) cell lines, efficient gRNAs for each gene target were first identified as described above. For the 6× KO cell pool, six gRNAs were pooled together, at a 1:1:1 ratio of crRNA (20 pmol) to tracrRNA (20 pmol) to Cas9 protein (20 pmol), to form 120 pmols of RNP which were transfected into twelve million cells three sequential times 72 hours apart between each transfection. For the 10× KO cell pool, a total of 4 sequential transfections were performed using ten gRNAs. For transfection rounds 1-3, nine gRNAs were pooled together at a 1:1:1 ratio of XT-gRNA (20 pmol) to tracrRNA (20 pmol) to Cas9 protein (20 pmol) with a total of 180 pmols of RNP transfected into twelve million cells. For the 4th round of transfection, the 10th gRNA targeting gene E was transfected at the same 1:1:1 ratio of XT-gRNA (20 pmol) to tracrRNA (20 pmol) to Cas9 protein (20 pmol), with 20 pmols of RNP transfected. Editing efficiency was measured after each transfection as described above.

The 6× and 10× cell KO pools were single-cell cloned by limiting dilution into 384-well plates with a target density of 0.4 cells/well. Plates were cultured for 2 weeks at 37° C., 5% CO₂, and 80% humidity, followed by automated confluency-based hit-picking and expansion to 96-well plates using Microlab STAR (Hamilton).

### Example 2: Identification of Efficient gRNA for Each of Each Target Gene

To identify a efficient gRNA for each target gene, transfections of purified Cas9 protein bound to synthetic gRNA in an RNP complex to simultaneously screen several gRNAs for a given gene were performed. For quantification of editing efficiencies, Inference of CRISPR Edits (ICE) was used, an online software for analyzing Sanger sequencing data (synthego.com/guide/how-to-use-crispr/ice-analysis-guide), which has been extensively validated for targeted NGS (Hsiau T, et al. Inference of CRISPR edits from Sanger trace data. BioRxiv. Published online 2018:251082.), to identify the type and quantitatively infer the abundance of Cas9 induced edits (Brinkman E K, et al., Easy quantitative assessment of genome editing by sequence trace decomposition. Nucleic acids research. 2014;42(22): e168-e168). The proposed workflow to accomplished transfecting cells with RNP, extracting DNA from the transfected cells, amplifying the region surrounding the gRNA cut sites, and analyzing the sequenced amplicon in only four days (FIG. 1A). This protocol allowed seamless and quick identification of highly efficient gRNAs from those with far lower editing efficiency. To illustrate the throughput of this protocol, three different gRNAs targeting gene A were individually transfected into CHO cells, alongside a gRNA targeting luciferase as a control. The gRNAs showed a wide range of indel efficiencies, with gRNA-3 showing the highest % indel (FIG. 1B), as determined by ICE software. ICE software aligns sequences of edited samples, comparing them to the control sample around the cut sites (vertical dotted line) to provide information on the type and abundance of indels (FIG. 1C). FIG. 1C top panel represents an example of a gRNA with extremely low editing efficiency (gRNA-1) where sequencing traces of the edited region is almost identical to the unedited control sequence. In contrast, FIG. 1C bottom panel represents a gRNA with high editing efficiency (gRNA-3) where the high level of convolution after the cut site for gRNA-3 suggests extensive editing. Furthermore, the ICE algorithm was able to deconvolute the edited trace in order to deduce the type of indels and % contribution at the target region (FIG. 1C, bottom panel).

To confirm that the indel efficiencies from ICE analysis correlated to a reduction in protein expression, two gRNAs targeting gene B were analyzed by Western blot analysis (FIG. 1D). As illustrated, indel efficiencies for gRNA-1 (9%) and gRNA-2 (65%) correlated very well to the observed band intensities of the target protein. Indel efficiencies from ICE analysis was also confirmed by TA cloning followed by sequencing of the individual PCR products from three different gRNA targets (genes C-E). As shown, TA cloning results largely correlated with % indel calculated by ICE analysis (FIG. 1E). Table 2 lists the efficient gRNAs identified for each gene target tested in the aforementioned experiments.

TABLE 2
Target Knockout Gene Specifications

| Represented | |
|---|---|
| gene name in | |
| the Figures | |
| 1-4 | *gRNA sequence |
| Gene A | TCCAAAACTCTA |
| | TCAAAACCGGG |
| Gene B | TCTTACCTCTGTA |
| | TTCACTTAGG |
| Gene C | GAAGCCTAAACT |
| | GATGTACCAGG |
| Gene D | CAGCAACACCTC |
| | AGTCAGCGAGG |
| Gene E | AGAGAGGTTCCG |
| | CCACACAAAGG |
| Gene F | ACCGAAATGATC |
| | AGGTACTGGGG |
| Gene G | CTGCTGTAACCC |
| | CATAAGCATGG |
| Gene H | GGAAGCCAAGAA |
| | GAAGAAGGAGG |
| Gene I | ATCCCGGGACAC |
| | AGACACAAAGG |
| Gene J | CAGAGTTTGACC |
| | GCCTCCCAAGG |
| Gene K | ATCCAGCAGTCA |
| | ATGATAACAGG |
| GFP | CAGCTTAGCACC |
| | TTCGGTCAGGG |

*5′ to 3′ strand with underlined PAM site

### Example 3: Optimization of RNP Transfection to Improve Knockout Efficiency

To improve knockout efficiency, varying levels of the total amount of transfected RNP was tested. Starting from a baseline of 20 pmol RNP per twelve million cells (1× concentration), and using multiples of 20 pmol in ratio to the same number of cells, GFP expressing host cells were transfected with 0.1× to 2× RNP targeting GFP protein expression. The percentage of GFP expressing cells was measured by flow cytometry three days after electroporation (FIG. 2A). While lowering the amount of transfected RNP reduced indel efficiency, increasing the amount of RNP did not substantially improve efficiency of this highly efficient gRNA.

Since Cas9 protein and gRNA are in equilibrium with the assembled RNP, it was tested whether increasing the gRNA concentration would improve the efficacy of RNP. Varying amounts of the cr/tracrRNA complex were annealed for two different targets, genes F and G, and transfected into cells with constant amounts of Cas9 protein. The data suggests that using excess sgRNA could modestly improve editing efficiency (FIG. 2B).

For intrinsically weak gRNAs, alternate types of gRNAs such as crRNA-XT (XT-gRNA) and sgRNA, have been reported to further increase gene editing efficiency (idtdna.com/pages/products/crispr-genome-editing/alt-r-crispr-cas9-system). crRNA is a two-part gRNA that requires annealing to tracrRNA; XT-gRNA is an extended half-life variant of crRNA produced by IDT, and sgRNA is full-length gRNA which can be directly complexed to Cas9 (idtdna.com/pages/products/crispr-genome-editing/alt-r-crispr-cas9-system). Versions of these gRNAs were synthesized targeting the same sequence of gene D and observed considerably higher indel efficiencies for either XT-gRNA or sgRNA (FIG. 2C).

In parallel, it was tested tested whether sequential rounds of transfection with the screened gRNAs could generate a final pool of cells with higher levels of simultaneous knockout efficiency for 6 target genes at once. Using six gRNAs (see Table 2) with varying levels of editing efficiency, equal amounts of crRNA/tracrRNA were pooled for each gene and mixed the annealed guides with Cas9 protein to form RNP. CHO cells were transfected with the RNP three sequential times, 72 hrs apart, and indel efficiency was measured after each round of transfection by PCR and ICE analysis. Sequential transfections had no impact on cell viability and for the most efficient gRNA (targeting gene E) multiple transfections did not affect the level of editing. However, the extent of editing was increased for the weaker gRNAs (targeting genes C, F, G, and K) after each round of transfection, reaching greater than 76% indels in the population (FIG. 2D). From this pool, single cell clones were generated and 496 clones were screened for knockouts and eight clones (1.61%) were identified to have complete knockouts of all 6 genes.

### Example 4: Isolation of Clones with Simultaneous Knockout of Up to Ten Genes Using Pools of Efficient gRNAs in a Multiplex Transfection

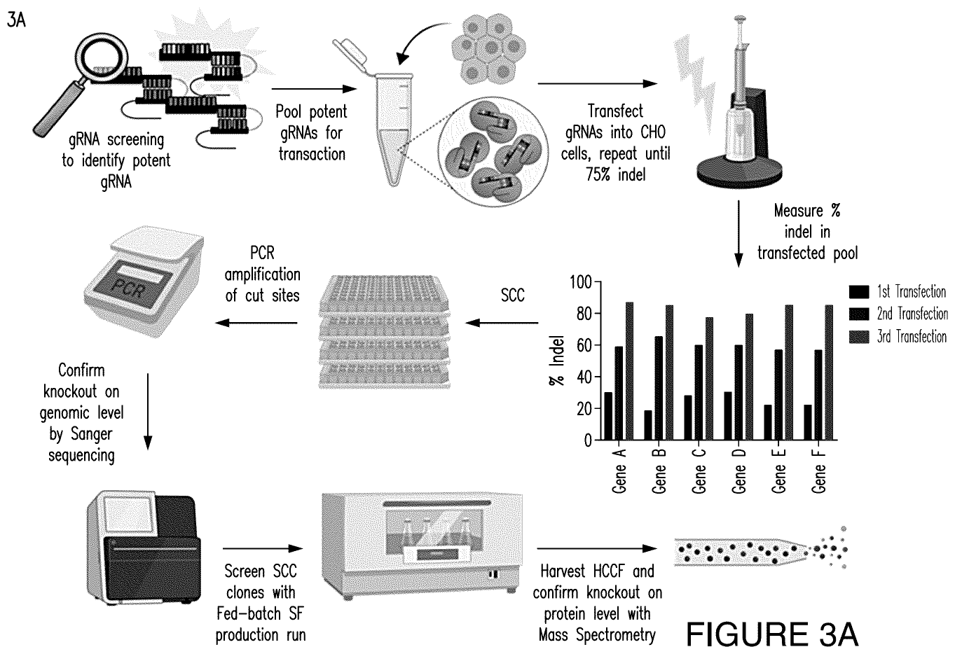

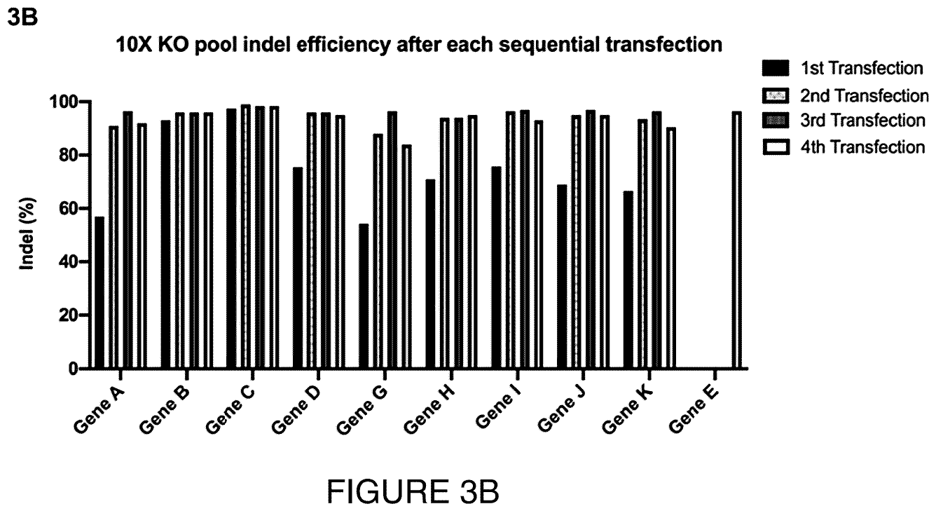

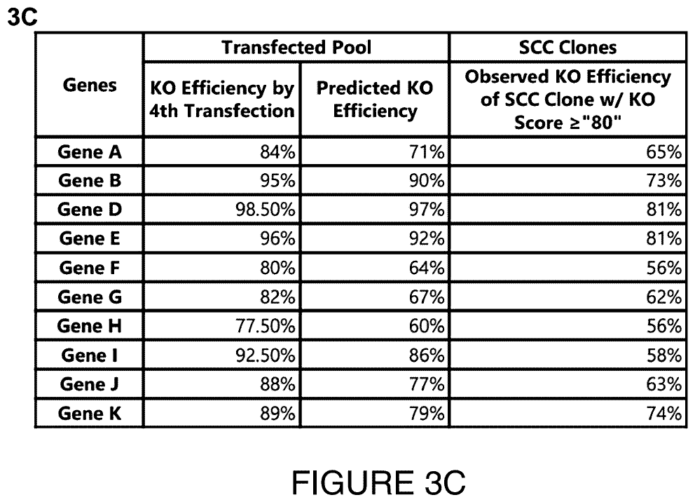

Combining all of optimizations steps to increase overall knockout efficiency, a workflow was streamlined for generating single cell clones from a pool in which ten genes (Table 2) were simultaneously knocked out (FIG. 3A). The strongest candidate gRNA for each target gene was identified (as in FIG. 1A) and crRNA-XT versions of these gRNAs were used to transfect cells four times sequentially. For the highly efficient gRNA targeting gene E only one round of transfection (in the last round) was performed. The data suggest that only two sequential transfections were sufficient to disrupt all ten genes with a minimum of 84% indel (FIG. 3B). Single cell cloning and the 10× knockout out pool followed by PCR screening, Sanger sequencing, and ICE analysis allowed prediction of knockout efficiencies for each of the target genes. Since the ICE knockout score represents only the subpopulation of cells that have either a frameshift indel or a fragment deletion (of 21+ bp), the knockout efficiencies were tabulated for each of the 10 target genes after the fourth transfection (FIG. 3C). Assuming all genes were present in two alleles in a single cell, the predicated knockout efficiency was calculated by squaring the pool knockout frequency. The observed knockout efficiency of single cell clones was calculated by counting the proportion of clones with an ICE knockout score cut off of ≥80%. This percentage was slightly lower than that of the predicted knockout efficiency in the transfected pool. This could be due to slightly lower survival of knockout clones from the single cell cloning process, or lower quality of high throughput PCR amplification and Sanger sequencing, or a small percentage of triploid or higher ploidy cells in the population. From the 704 single cell clones screened, six were found to have genomic DNA level knockout of all ten genes, corresponding to a 0.9% probability. As the number of targets was increased, a larger number of clones needed to be screened since the probability of obtaining a complete knockout clone was expected to be lower.

### Example 5: Multiplex Knockout Cell Lines Displayed Comparable Growth Characteristics to that of the Wildtype

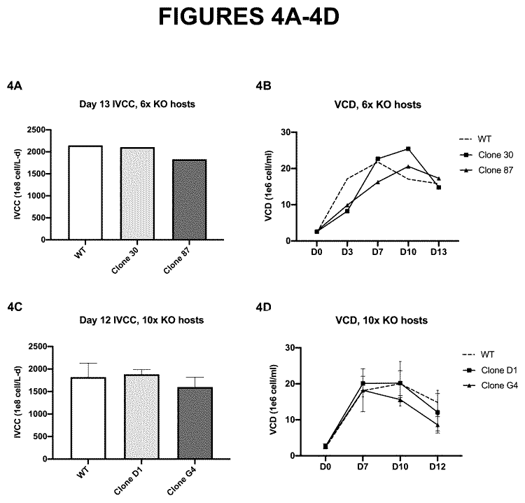

To confirm knockout on the protein level, the clones were scaled up, a fed-batch production culture was conducted, and the harvested cell culture fluid was analyzed by LC-MS/MS. Proteins from all ten genes were identified in the wildtype CHO cell HCCF but not in any of the knockout clones, confirming their absence at the protein level. Following the identification of 6× KO or 10× KO SCC clones, two clones from each arm were subjected to a shake flask fed-batch production evaluation to compare their growth to the wildtype parental control. For both 6× KO (clones 30 and 87) and 10× KO (clones D1 and G4) arms, the KO clones had comparable cell growth to the parental cell line as indicated by integrated variable cell count (IVCC) and viable cell density (VCD) measurements (FIGS. 4A-4D).

The contents of all figures and all references, patents and published patent applications and Accession numbers cited throughout this application are expressly incorporated herein by reference.

What is claimed is:

1. A method of producing a cell comprising edits at two or more target loci:
(a) combining two or more guide RNAs (gRNAs) capable of directing CRISPR/Cas9-mediated indel formation at respective target loci with Cas9 protein to form a ribonucleoprotein complex (RNP);
(b) serially transfecting a population of cells with the RNP until at least about 10% indel formation is achieved at each target locus; and
(c) isolating a cell comprising edits at two or more target loci by single cell cloning of the cell from the population of serially transfected cells.

2. The method of claim 1, wherein the population of cells is serially transfected with the RNP until at least about 20% indel formation is achieved at each target locus.

3. The method of claim 1, wherein the ratio of moles of RNP to number of transfected cells is between about 0.1 pmol per 10⁶ cells to about 5 pmol per 10⁶ cells

4. The method of claim 1, wherein three or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

5. The method of claim 1, wherein the cell is a T cell, an NK cell, a B cell, a dendritic cell, a CHO cell, a COS-7 cell; an HEK 293 cell, a BHK cells, a TM4 cell, a CV1 cell; a VERO-76 cell; a HELA cells; or an MDCK cell.

6. The method of claim 1, wherein the two or more gRNAs capable of directing CRISPR/Cas9-mediated indel formation at respective target loci are identified via a efficiency screen comprising:
(a) transfecting a population of cells with a population of RNPs, where each RNP comprises a gRNA capable of directing CRISPR/Cas9-mediated indel formation at a target locus; and
(b) sequencing the target loci to identify gRNAs based on their efficiency in directing CRISPR/Cas9-mediated indel formation.

7. A host cell composition, wherein the host cell comprises:
(a) a nucleic acid encoding a non-endogenous polypeptide of interest; and
(b) edits at two more target loci, wherein the edits are the result of:
i. combining two or more gRNAs capable of directing CRISPR/Cas9-mediated indel formation at respective target loci with Cas9 protein to form an RNP;
ii. serially transfecting a population of cells with the RNP until at least about 10% indel formation is achieved at each target locus; and
iii. isolating the host cell comprising edits at two or more target loci by single cell cloning of the host cell from the population of serially transfected cells.

8. The host cell composition of claim 7, wherein the population of cells is serially transfected with the RNP until at least about 20% indel formation is achieved at each target locus.

9. The host cell composition of claim 7, wherein the ratio of moles of RNP to number of transfected cells is between about 0.1 pmol per 10⁶ cells to about 5 pmol per 10⁶ cells

10. The host cell composition of claim 7, wherein three or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

11. The host cell composition of claim of claim 7, wherein the host cell is a T cell, an NK cell, a B cell, a dendritic cell, a CHO cell, a COS-7 cell; an HEK 293 cell, a BHK cells, a TM4 cell, a CV1 cell; a VERO-76 cell; a HELA cells; or an MDCK cell.

12. The host cell composition of claim 7, wherein the two or more gRNAs capable of directing CRISPR/Cas9-mediated indel formation at respective target loci are identified via an efficiency screen comprising:
(a) transfecting a population of cells with a population of RNPs, where each RNP comprises a gRNA capable of directing CRISPR/Cas9-mediated indel formation at a target locus; and
(b) sequencing the target loci to identify gRNAs based on their efficiency in directing CRISPR/Cas9-mediated indel formation.

13. The host cell composition of claim 7, wherein polypeptide of interest comprises an antibody or an antigen-binding fragment thereof.

14. The host cell composition of claim 13, wherein the antibody is a multispecific antibody or an antigen-binding fragment thereof.

15. The host cell composition of claim 13, wherein the antibody is a chimeric antibody, a human antibody or a humanized antibody.

16. The host cell composition of claim 13, wherein the antibody is a monoclonal antibody.

17. A method producing a polypeptide of interest comprising:
(a) culturing a host cell composition comprising:
i. a nucleic acid encoding a non-endogenous polypeptide of interest; and
ii. edits at two or more target loci, wherein the edits are the result of:
1. combining two or more gRNAs capable of directing CRISPR/Cas9-mediated indel formation at respective target loci with Cas9 protein to form an RNP;
2. serially transfecting a population of cells with the RNP until about 10% indel formation is achieved at each target locus; and
3. isolating the host cell comprising edits at two or more target loci by single cell cloning of the host cell from the population of serially transfected cells; and
(b) isolating the polypeptide of interest expressed by the cultured host cell.

18. The method of claim 17, wherein the population of cells is serially transfected with the RNP until at least about 20% indel formation is achieved at each target locus.

19. The method of claim 17, wherein the ratio of moles of RNP to number of transfected cells is between about 0.1 pmol per 10⁶ cells to about 5 pmol per 10⁶ cells.

20. The method of claim 17, wherein three or more gRNAs capable of directing CRISPR/Cas9-mediated indel formation at respective target loci are combined with Cas9 protein to produce RNPs and the RNPs are serially transfecting into a population of cells until at least about 10% indel formation is achieved at each target locus.

21. The method of any one of claims 20, wherein the RNPs are serially transfecting into a population of cells until at least about 20% indel formation is achieved at each target locus.

22. The method of claim 17, wherein the cell is a T cell, an NK cell, a B cell, a dendritic cell, a CHO cell, a COS-7 cell; an HEK 293 cell, a BHK cells, a TM4 cell, a CV1 cell; a VERO-76 cell; a HELA cells; or an MDCK cell.

23. The method of claim 17, wherein the two or more gRNAs capable of directing CRISPR/Cas9-mediated indel formation at respective target loci are identified via a efficiency screen comprising:
(a) transfecting a population of cells with a population of RNPs, where each RNP comprises a gRNA capable of directing CRISPR/Cas9-mediated indel formation at a target locus; and
(b) sequencing the target loci to identify gRNAs based on their efficiency in directing CRISPR/Cas9-mediated indel formation.

24. The method of claim 17, wherein the method comprises purifying the product of interest, harvesting the product of interest, and/or formulating the product of interest.

25. The method of claim 17, wherein the cell is a mammalian cell.

26. The method of claim 25, wherein the mammalian cell is a CHO cell.

27. The method of claim 17, wherein polypeptide of interest comprises an antibody or an antigen-binding fragment thereof.

28. The method of claim 27, wherein the antibody is a multispecific antibody or an antigen-binding fragment thereof.

29. The method of claim 27, wherein the antibody is a chimeric antibody, a human antibody or a humanized antibody.

30. The method of claim 27, wherein the antibody is a monoclonal antibody.
